(19) United States
(12) Patent Application Publication
(10) Pub. No.: US 2025/0097304 A1
(43) Pub. Date: Mar. 20, 2025

(54) SESSION MANAGEMENT FOR VARIABLE-LENGTH MESSAGE STREAMS

(71) Applicant: Oracle International Corporation, Redwood Shores, CA (US)

(72) Inventors: Syed Najeeb Andrabi, Cupertino, CA (US); Sachin Goel, Bothell, WA (US)

(73) Assignee: Oracle International Corporation, Redwood Shores, CA (US)

(21) Appl. No.: 18/829,915

(22) Filed: Sep. 10, 2024

Related U.S. Application Data
(60) Provisional application No. 63/583,230, filed on Sep. 15, 2023.

(51) Int. Cl. H04L 67/146; H04L 67/63

(52) U.S. Cl. CPC H04L 67/146; H04L 67/63

(57) ABSTRACT

Techniques are disclosed for session management for variable-length message streams. In an example method, a computing system establishes a first session by receiving, from a first computer system, registration information including a first session identifier and a specification of a channel; determining a stream orchestration instance for the channel; and joining the first computer system to the first session for the stream orchestration instance based on the first session identifier. The computing system receives, from a second computer system, a message including context information, the context information including the first session identifier. The computing system identifies the first session based on the first session identifier, the first session having one or more member computer systems. The computing system outputs the message to at least one of the one or more member computer systems of the first session.

### CROSS-REFERENCE TO RELATED APPLICATIONS

The present application claims the benefit of and priority to U.S. Provisional Application No. 63/583,230, filed Sep. 15, 2023 and entitled “CLINICAL DIGITAL ASSISTANT,” the entire contents of which are incorporated herein by reference for all purposes.

### BACKGROUND

Clinical environments such as healthcare facilities often include different healthcare providers working together and communicating with one another to treat patients. Documenting patient encounters, capturing information conveyed during those encounters and/or pertaining to events occurring before and/or after the encounters, populating patient records such as electronic health records, and healthcare practice management are integral parts of the practice of many healthcare providers and important to ensuring high-quality healthcare. Traditional means for performing tasks associated with providing healthcare often involve several different devices such as listening devices, portable electronic devices, workstations, and the like and end users who are equipped with the training, knowledge, experience, and skills to properly utilize these devices and participate in the healthcare process. Relying on different devices and qualified end users to perform clinical tasks is cumbersome, time and resource intensive, costly, and reduces efficiencies, which may lead to lower-quality healthcare.

### BRIEF SUMMARY

Techniques are disclosed herein for session management for variable-length message streams. Stream orchestration can be provided by way of routes specified using an implementation-independent “polyglot” stream orchestration language (SOL). Session management for groups of related consumers can be provided. An example of a group of related consumers is a team of healthcare providers collaborating to diagnose a patient, each provider using one or more client devices. Session management can be used in this example to output messages from a backend services to each of the client devices is used by the team of healthcare providers.

In some embodiments, a computer-implemented method includes establishing a first session, wherein establishing the first session includes receiving, from a first computer system, registration information, the registration information including a first session identifier and a specification of a channel; determining a stream orchestration instance for the channel; and joining the first computer system to the first session for the stream orchestration instance based on the first session identifier. The computer-implemented method further includes receiving, from a second computer system, a message, the message including context information, the context information including the first session identifier. The computer-implemented method further includes identifying the first session based on the first session identifier, the first session having one or more member computer systems. The computer-implemented method further includes outputting the message to at least one of the one or more member computer systems of the first session.

In some embodiments, the message is a variable-length message further including a payload. The message is encoded in a binary format and the context information comprises a context length and one or more key-value pairs, the context length defining a demarcation between the context information and the payload, the context information and the payload both characterized by a variable-length.

In some embodiments, the computer-implemented method further includes determining, from the context information, routing information including a specification of the at least one of the one or more member computer systems, the routing information based on a set of stream orchestration specifications, wherein each stream orchestration specification of the set of stream orchestration specifications includes one or more tasks defining a route, including at least one routing task and an output task, the output task including the specification of the at least one of the one or more member computer systems.

In some embodiments, the routing information includes one or more first tasks defining a first route and the routing information is determined based on a specification of the first route that is included in the context information.

In some embodiments, at least one of the one or more member computer systems is a clinical transcription service, an ambient listening service, or a clinical automation service and outputting the message to the at least one of the one or more member computer systems causes an update to a clinical record.

In some embodiments, joining the first computer system to the first session for the stream orchestration instance based on the first session identifier includes generating a new session for the stream orchestration instance.

In some embodiments, joining the first computer system to the first session for the stream orchestration instance based on the first session identifier includes joining an existing session for the stream orchestration instance.

In some embodiments, the computer-implemented method further includes determining that the first session for the stream orchestration instance has expired and generating a new session for the stream orchestration instance.

In some embodiments, the computer-implemented method further includes, responsive to receiving the registration information, generating the stream orchestration instance.

In some embodiments, the at least one of the one or more member computer systems includes the first computer system and computer-implemented method further includes receiving, from the first computer system, a second message, the second message including second context information, the second context information including the first session identifier. The computer-implemented method further includes identifying the first session based on the first session identifier. The computer-implemented method further includes outputting the message to the at least one of the one or more member computer systems of the first session including the second computer system.

In some embodiments, the computer-implemented method further includes receiving, from the first computer system, a second message, the second message including second context information, the second context information including the first session identifier. The computer-implemented method further includes identifying the first session based on the first session identifier. The computer-implemented method further includes outputting the message to the at least one of the one or more member computer systems of the first session not including the second computer system.

In some embodiments, the message is based on a format described using a specification written in a capability interface definition language.

In some embodiments, the channel is defined using the capability interface definition language.

In some embodiments, the capability interface definition language is AsyncAPI and the message is received using a WebSocket endpoint.

In some embodiments, the context information includes a plurality of sections, wherein content of each section is defined in the specification using the capability interface definition language.

Some embodiments include a system that includes one or more processing systems and one or more computer-readable media storing instructions which, when executed by the one or more processing systems, cause the system to perform part or all of the operations and/or methods disclosed herein.

Some embodiments include one or more non-transitory computer-readable media storing instructions which, when executed by one or more processing systems, cause a system to perform part or all of the operations and/or methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

Features, embodiments, and advantages of the present disclosure are better understood when the following Detailed Description is read with reference to the accompanying drawings.

FIG. 1 shows an example AI-enabled system for providing a clinical digital assistant, according to some examples of the present disclosure.

FIG. 2 depicts an example system for stream orchestration for variable-length message streams, according to some examples of the present disclosure.

FIG. 3 depicts another example system for stream orchestration for variable-length message streams, according to some examples of the present disclosure.

FIGS. 4A and 4B depict example messages that may be used for stream orchestration for variable-length message streams, according to some examples of the present disclosure.

FIGS. 5A-5C depict example routes that may be used in some implementations of systems for stream orchestration for variable-length message streams, according to some examples of the present disclosure.

FIG. 6 depicts an example stream orchestration specification using an example SOL for stream orchestration for variable-length message streams, according to some examples of the present disclosure.

FIG. 7 depicts another example system for stream orchestration for variable-length message streams, according to some examples of the present disclosure.

FIG. 8 is a process flow for stream orchestration for variable-length message streams, according to some examples of the present disclosure.

FIG. 9 is a process flow for stream orchestration for variable-length message streams, according to some examples of the present disclosure.

FIG. 10 is a block diagram illustrating one pattern for implementing a cloud infrastructure as a service system, according to at least one embodiment.

FIG. 11 is a block diagram illustrating another pattern for implementing a cloud infrastructure as a service system, according to at least one embodiment.

FIG. 12 is a block diagram illustrating another pattern for implementing a cloud infrastructure as a service system, according to at least one embodiment.

FIG. 13 is a block diagram illustrating another pattern for implementing a cloud infrastructure as a service system, according to at least one embodiment.

FIG. 14 is a block diagram illustrating an example computer system, according to at least one embodiment.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

Artificial intelligence (AI) and other modern technologies have opened new doors for efficient and effective delivery of healthcare. One new approach for providing healthcare that has become prevalent as of late involves digital assistants (also referred to as bots, chatbots, chatterbots, talkbots, skillbots). A digital assistant is an AI-based tool that can converse with end users. Generally, a digital assistant can respond to natural-language messages (e.g., questions or comments) provided by an end user using an application incorporates the bot. One example application is a messaging application in which natural-language messages are exchanged. End users interact with the digital assistant through conversational interactions (sometimes referred to as a conversational user interface (UI)), just as end users interact with other people. End users also interact with the bot through other types of interactions, such as transactional interactions (e.g., with a banking bot that is at least trained to transfer money from one account to another), informational interactions (e.g., with a human resources bot that is at least trained check the remaining vacation hours the user has), and/or retail interactions (e.g., with a retail bot that is at least trained for discussing returning purchased goods or seeking technical support).

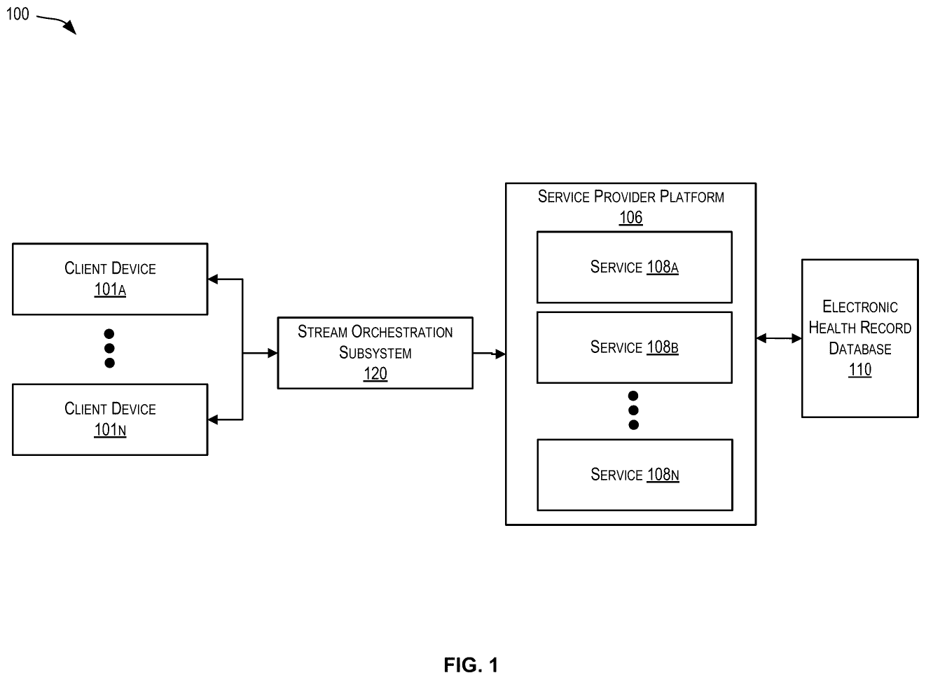

A digital assistant can involve several different client devices interfacing with several different services or other downstream computing devices. FIG. 1 shows an example AI-enabled system 100 for providing a clinical digital assistant, according to some examples of the present disclosure. As shown in FIG. 1, system 100 includes client devices 101a . . . n, a service provider platform 106, and electronic health record database 110. AI-enabled system 100 provides intelligent assistant services to healthcare providers such as doctors, nurses, technicians, clinicians, medical personnel, and the like. As used herein, the term healthcare provider generally refers to healthcare practitioners and professionals including, but not limited to: physicians (e.g., general practitioners, specialists, surgeons, etc.); nurse professionals (e.g., nurse practitioners, physician assistants, nursing staff, registered nurses, licensed practical nurses, etc.); other professionals (e.g., pharmacists, therapists, technicians, technologists, pathologists, dietitians, nutritionists, emergency medical technicians, psychiatrists, psychologists, counselors, dentists, orthodontists, hygienists, etc.).

The client devices 101a . . . n may include devices executing suitable client software such as mobile devices, tablets, laptop, desktops, smartwatches, and so on. Healthcare providers can interact with the client devices 101a . . . n based on touch input (e.g., tapping, swiping, pinching) and voice input captured by the electronic device, etc. Healthcare providers can use the client devices 101a . . . n to obtain information about a patient such as medical information from an electronic health record for the patient stored in the electronic health record database 110. Healthcare providers can also use the client devices 101a . . . n to collect information about a patient and information relevant to the observation, care, treatment, and/or management of a patient. Client devices 101a . . . n may include data collection capabilities including the capability to collect ambient sounds (natural and/or artificial) and information about events occurring in an environment in which the mobile application is located. Some client devices 101a . . . n can be used together to provide a multi-modal user experience. In other implementations, various client devices 101a . . . n can provide overlapping or different functions.

The client devices 101a . . . n can be connected to a service provider platform 106, via steam orchestration subsystem 120, which can provide assistant services to the client devices 101a . . . n. Services 108a . . . n can include, but are not limited to, authentication services, user management services, frontend services (e.g., entry point (facade) to all services), and other management services. Services 108a . . . n can also include, but are not limited to, ambient services (e.g., an AI-powered, voice-enabled service that automatically documents patient encounters accurately and efficiently at the point of care and provides quick action suggestions), dictation services (e.g., a service that allows doctors to generate medical records from voice e.g., using a Large Language Model (LLM) or pre-seeded templates), digital assistant services (e.g., a server that allows you to create and deploy chatbots), speech services (e.g., an AI service that applies Automatic Speech Recognition (ASR) technology to transform audio-based content into text). The client devices 101a . . . n along with the service provider platform 106 can enable clinicians to obtain information relevant to their patients (e.g., information in stored in electronic health record database 110) faster along with placing orders fasters (e.g., tests, medications, laboratories) all using a conversational experience (e.g., using one or more voice interfaces).

Multiplexed networked communication between the client devices 101a . . . nand the services 108a . . . n can present a formidable challenge, particularly at large scale. In a production setting, high-bandwidth, streaming data such as video or audio data may be sent continuously from the client devices 101a . . . n to multiple downstream consumer services 108a . . . n. In turn, the services 108a . . . n may be continuously sending responses back to the client devices 101a . . . n. For example, a particular client device (e.g., a smartphone) used for dictation may stream binary audio data to a storage service, an ASR service, and an audio processing service. The particular client device may, in turn, receive transcribed speech from the ASR service in near-real-time or with minimized latency.

A large healthcare organization (e.g., a large city hospital or geographically dispersed hospital system) may have thousands, tens of thousands, or hundreds of thousands—or even more-client devices being used simultaneously for healthcare delivery in various capacities. The relatively smaller number of services 108a . . . n may likewise be sending data back to the large number of client devices. Handling the routing of data at this scale can quickly become intractable. Consequently, some systems may include a messaging or stream orchestration layer in between the client devices 101a . . . n and the services 108a . . . n to coordinate the sending and receiving of streamed data.

Existing approaches may involve a message broker or stream orchestration layer that can receive messages from client devices 101a . . . n or services 108a . . . n in a fixed, predefined format and route the messages to various channels using a queuing or publish/subscribe (pub/sub) mechanism. For example, existing stream orchestration layers may be implemented as an application written in a particular programming language, such as Java. The Java program code may define a number of endpoints for receiving messages and may include instructions for routing the messages between the client devices 101a . . . n and various services 108a . . . n. Existing systems thus implemented are typically stateless and route messages in accordance with predefined labels or rules.

These existing approaches lack the flexibility and capabilities needed to efficiently coordinate the sending and receiving of streamed data among the client devices 101a . . . n and the services 108a . . . n. Because existing approaches rely on fixed-length messages, the achievable efficiency is naturally limited. For example, a fixed-length message format may require padding or truncation of data, leading to inefficient use of bandwidth and increased transmission times. Similarly, existing systems that are coupled to a single programming language may have a limited ability to handle the diverse spectrum of data formats and protocols, complicating integration between the client devices 101a . . . n and the services 108a . . . n and limiting interoperability. Modification of such systems can involve a time-consuming process of rewriting, recompiling, and redeploying program code. Additionally, the stateless architectures used in existing systems means that message state, including useful abstractions such as sessions for groups of related client devices 101a . . . n, must be managed by the application. This is often done using a dedicated session management service, introducing additional complexity and potential points of failure or error. Such approaches are also slower and have increased bandwidth requirements, since session lookup may be needed every time a message is routed.

The developed approach described herein addresses these challenges and others by providing techniques for stream orchestration for variable-length message streams. In some examples, a stream orchestration subsystem 120 is provided that includes components for receiving and routing messages to and from client devices to various backend services. The stream orchestration subsystem 120 can receive variable-length messages in accordance with a predefined interface. The use of variable-length messages can provide optimized flexibility and efficiency through a binary, variable-length message specification that includes context information as encoded text in a semi-structured format and arbitrary-length binary content. Arbitrary length is enabled by including the length of the context information which can act as demarcation between the message context information and the actual content. The routes can be configured using a polyglot stream orchestration language (SOL) that is agnostic to the details of the underlying implementation of the routing tasks or destinations and can easily be modified or dynamically specified. The polyglot SOL further enables message publishers and consumers to dynamically specify routes or orchestrations using the SOL. The stream orchestration subsystem 120 can likewise publish orchestration capabilities to producers and consumer using the SOL. The SOL can provide increased flexibility through constructs that enable routing steps to be performed sequentially, in parallel, or conditionally. Additionally, the SOL provides variables representing various states that can be used to further improve flexibility and efficiency. The stream orchestration subsystem 120 can further include a session management component that can, for example, be used to ensure that messages are routed to all client devices in use by a particular user or users, thereby directing messages to multiple client devices for a single user or users using a specified route. The session management component can enable complex interactions between clients and services at the message routing layer rather than by application code.

The following examples are provided to illustrate certain concepts. In various embodiments, a computer-implemented method includes receiving, by a computer system including a stream orchestration subsystem 120, a variable-length message in which the variable-length message includes context information and a payload. For example, consider an example client device used by a healthcare provider for applications such as dictation, automated note generation, clinical assistance, or other related application. The client device may use various backend services such as ASR, persistent storage, translation, or a note generation service. To do so, the client device outputs a number of variable-length messages to the services.

For instance, the example client device may use an ASR service to transcribe the healthcare provider's voice to text. The client device can output the provider's voice as a stream of variable-length messages. Each message can include context information and a payload. The context information can include information about the provider, the patient, the encounter, and so on. The context information may also include a sequence number or other information used to order and aggregate the audio data. The context information may further include an indication of the length of the context information, acting as a line of demarcation between the context information and the payload. The payload may be a portion of the audio data.

The stream orchestration subsystem 120 then determines, from the context information, routing information that identifies at least one consumer of the variable-length message. In this example, the consumer is the ASR service. The routing information may determined using the context information based on a set of stream orchestration specifications written in a SOL that is agnostic to the underlying implementation details of the stream orchestration subsystem 120. For example, the routing information may include a number of routes, each route including a number of tasks to be executed sequentially or in parallel by the stream orchestration subsystem 120. The context information may explicitly identify a route, or a route may be determined dynamically using, for example, the context information. In this example, static routing is used. In static routing, a route to the ASR service may be explicitly identified in the context information of the received variable-length message.

The stream orchestration subsystem 120 then outputs the variable-length message to the consumer. For example, a variable-length message, including a portion of the audio data for transcription by the ASR service, may be routed through a series of tasks including an output task. The output task can cause the variable-length message to be output the ASR service using a suitable messaging protocol.

In various embodiments, another computer-implemented method involves establishing a session by a computer system including a stream orchestration subsystem 120. Establishing the session may include first receiving, from a client device, registration information. The registration information may be received at, for example, a representational state transfer (REST) application programming interface (API) endpoint or at a WebSocket endpoint. The registration information may include a session identifier and a specification of a channel. The registration information can be used to create a new session or to join an existing session. Likewise, the registration information can be used to “subscribe” to the channel to receive messages that are published to the channel. Conversely, the registration information can be used to register as a “publisher” to the channel.

To establish the session, the stream orchestration subsystem 120 then determines a stream orchestration instance for the channel or “channel instance.” The stream orchestration instance can be identified from among a collection of already existing instances, or a new instance can be instantiated. The stream orchestration instance may include dedicated set of components (e.g., a class instance) for processing inbound messages, branching or splitting message streams according to certain conditions, or outputting messages to downstream consumer services. The stream orchestration instance may be associated with a particular session such that its components serve only the client devices associated with that session. The stream orchestration subsystem 120 can then join the client device to the determined stream orchestration instance based on the session identifier. For example, information associating the client device with the determined stream orchestration instance (e.g., a lookup or hash table) can be ephemerally or persistently stored for use during routing of messages.

The stream orchestration subsystem 120 then receives, from a service, a message. For example, the message may include textual audio data transcribed by an ASR service. The message may include context information, such as information that can be used to identify a route to one or more client devices, including the client device from the previous paragraph, as well as the session identifier used to determine the session. The stream orchestration subsystem 120 identifies the session based on the session identifier. For example, the lookup table updated above can be consulted to determine the appropriate session. Once the session is identified, the stream orchestration instance associated with the identified session can be used to output the message to the client device as well as all the other client devices associated with the session.

As used herein, when an action is “based on” something, this means the action is based at least in part on at least a part of the something. As used herein, the terms “similarly,” “substantially,” “approximately” and “about” are defined as being largely but not necessarily wholly what is specified (and include wholly what is specified) as understood by one of ordinary skill in the art. In any disclosed embodiment, the term “similarly,” “substantially,” “approximately,” or “about” may be substituted with “within [a percentage] of” what is specified, where the percentage includes 0.1, 1, 5, and 10 percent.

### Stream Orchestration for Variable-Length Message Streams

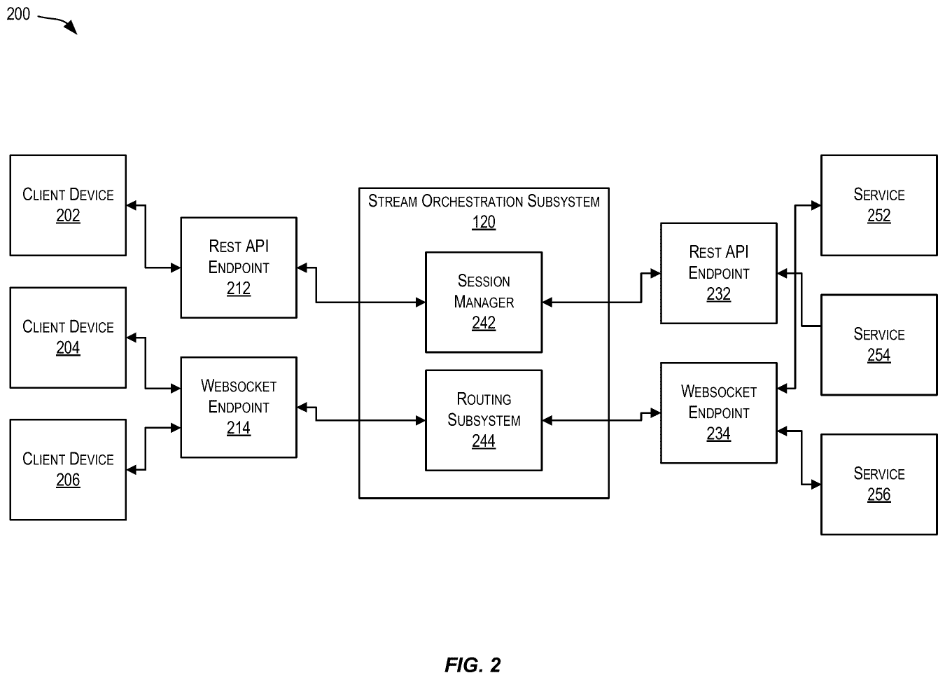

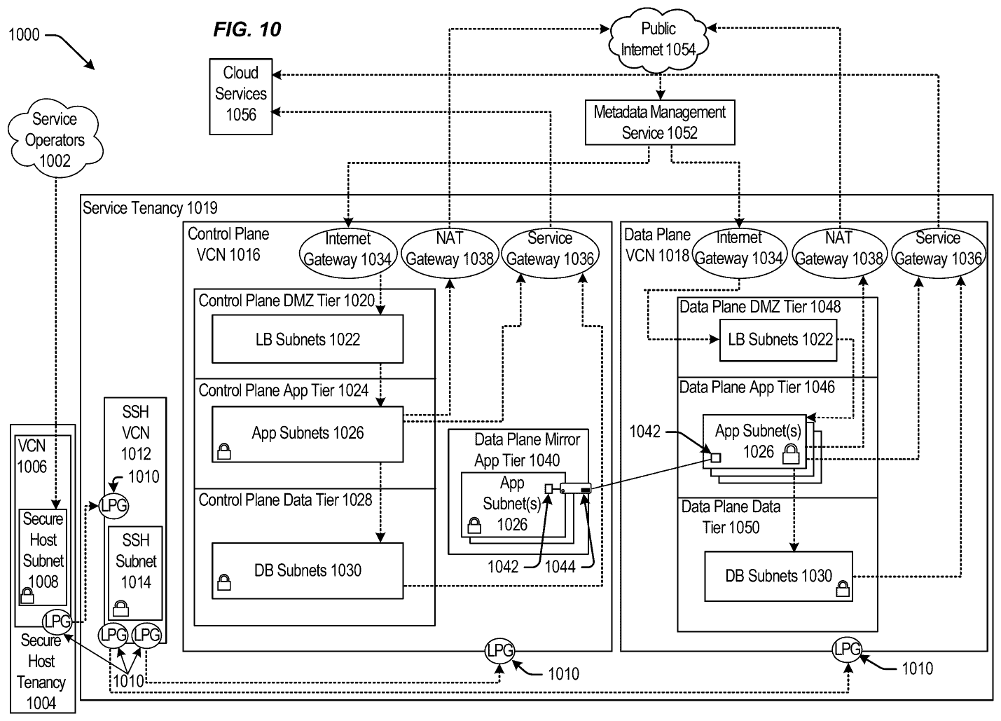

Some examples of systems and methods for stream orchestration for variable-length message streams disclosed herein can be used in the context of a cloud computing environment as part of an infrastructure as a service (IaaS) platform. Some of the components described herein may be deployed to one or more networked virtual cloud computing devices and may be provided to users, organizations, tenants, etc. as part of a collection of services provided to cloud computing subscribers. Examples of systems and methods for stream orchestration for variable-length message streams are shown in FIGS. 2-9. Detailed examples of IaaS systems that can support deployments of systems or components for stream orchestration for variable-length message streams are shown in FIGS. 10-14.

As shown above in FIG. 1, a stream orchestration subsystem 120 can be used to provide flexible and efficient message routing between or among client devices 101a . . . nand services 108a . . . n as part of a digital assistant implementation in the healthcare context as well as for many other applications. The stream orchestration subsystem 120 can receive variable-length messages and route them in accordance with routes specified using a polyglot SOL. The stream orchestration subsystem 120 can further provide session management for routing messages to groups of related consumers during the routing process, without the need for external session management by a standalone component or in the application layer. Examples of certain implementations are described in detail below to further illustrate some of these concepts.

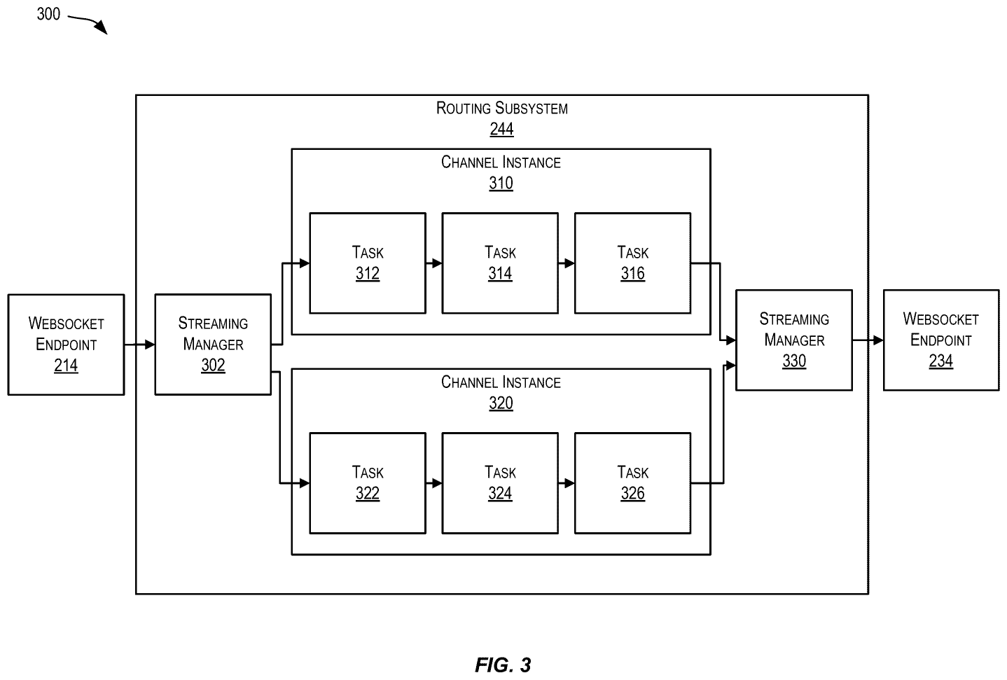

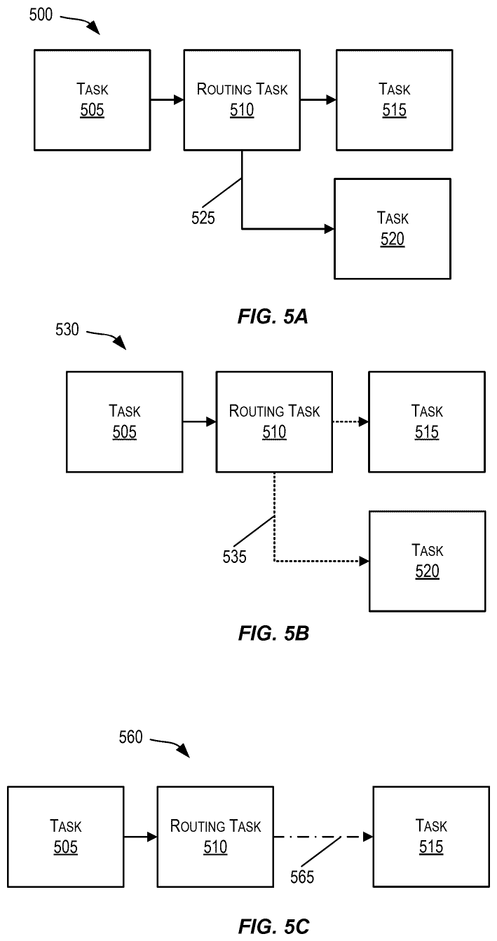

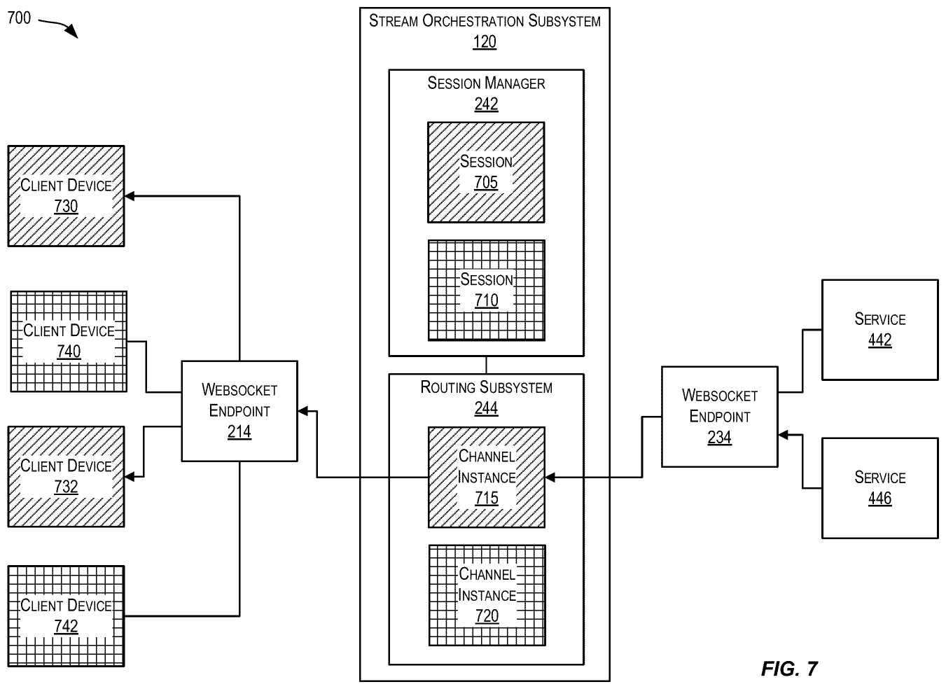

Turning first to FIG. 2, FIG. 2 depicts an example system 200 for stream orchestration for variable-length message streams, according to some examples of the present disclosure. System 200 shows a detail view of an example implementation of the stream orchestration subsystem 120 shown in example system 100. The stream orchestration subsystem 120 includes routing subsystem 244 and session manager 242. The routing subsystem 244 includes components for determining routing information based on the context information included with variable-length messages. The routing subsystem 244 also includes components that implement the routes explicitly or implicitly identified in the routing information in accordance with the polyglot SOL. An example implementation of the routing subsystem 244 is shown in FIG. 3 and several example routes are shown in FIGS. 5A-5C below. The stream orchestration subsystem 120 also includes session manager 242, which includes components for initiating and coordinating sessions for groups of related message consumers. A detailed description of one example implementation of session manager 242 is shown below in FIG. 7.

System 200 includes three client devices 202, 204, and 206. The client devices 202, 204, and 206 may be three particular client devices among numerous client devices 101a . . . n in use in a production system, shown to illustrate certain concepts. The client devices 202, 204, and 206 may be, for example, devices used by healthcare providers during clinical encounters for providing applications such as digital assistants, dictation or transcription, note generation, and so on. Such applications may utilize backend services 252, 254, and 256. Consequently, high volumes of messages may be output by the client devices 202, 204, and 206 to the services 252, 254, and 256, and then from the services 252, 254, and 256 back to the client devices 202, 204, and 206. In a simple example, a client device 202 may output audio data to service 252 to be transcribed. The transcribed audio data may later be output by the service 252 back to the client device 202.

Client device 202 is shown establishing a new session using REST API endpoint 212. For example, client device 202 may send a Hypertext Transfer Protocol (HTTP) request to REST API endpoint 212 including registration information using a GET or POST request to a suitable resource. The registration information may include a session identifier and a specified channel in the body, headers, or other parameters of the HTTP request. The session identifier can be an indication to create a new session or to join an existing session.

The HTTP request may include an indication to subscribe to the specified channel, register as a publisher to the specified channel, or both. A channel, as used herein, may refer generally to a collection of possible routes associated with one or more downstream consumers. For instance, a channel may be a collection of routes associated with a transcription service that converts spoken audio into a text transcript using ASR. The client device 202 may subscribe to the channel, which indicates an intent to receive messages from the consumer associated with the channel. The client device 202 may register as a publisher to the channel, which indicates an intent to send messages to the consumer associated with the channel.

In some examples, the client device 202 can establish the new session using WebSocket endpoint 214. For example, the client device 202 can output an authentication message using the WebSocket protocol to WebSocket endpoint 214 to create or join a session. Similarly to establishing a new session using the REST API endpoint 212, the authentication message can include registration information such as a session identifier and a specified channel in the initial WebSocket handshake, the query parameters of the connection URL, or as part of the first message sent after the WebSocket is established. The WebSocket authentication message may include an indication to subscribe to the specified channel, register as a publisher to the specified channel, or both. Use of the REST API endpoint 212 or WebSocket endpoint 214 for session management can be used individually or in combination. For example, a client device 202 can first establish a session using WebSocket endpoint 214 and then later update subscriptions using the REST API endpoint 212.

Upon receipt of the registration information and specified channel, the session manager 242 can determine a stream orchestration instance or “channel instance” for the channel. The stream orchestration instance may include the particular components that will be used to receive and send messages between the particular client device 202 (or client devices) and designated consumer, such as a service. The stream orchestration instance may be, for instance, class instances or objects initialized with information about the client device 202. However, in some examples, a stream orchestration instance may be initialized or later configured for use with multiple client devices and/or services. A particular channel can have numerous associated channel instances, each channel instance associated with a particular session, each session associated with one or more client devices and/or one or more services. Once the stream orchestration instance is initialized or identified, the session manager 242 can “join” the client device to the session by, for example, providing information about the channel instance to the client device 202 in a reply sent from the REST API endpoint 212 or WebSocket endpoint 214 to the registration request. In some examples, a session identifier such as an alphanumeric string can be used to identify the particular channel instance and associated session.

Also shown is service 254 establishing a session by registering using REST API endpoint 232 or WebSocket endpoint 234. Like the client device 202, the service 254 can subscribe to or register as a publisher to a particular channel. For example, during startup and initialization of service 254, the service 254 may output an HTTP request to REST API endpoint 232 to register as a subscriber and publisher for a particular channel or channels. In another examples, the service 254 can output a WebSocket authentication message to WebSocket endpoint 234 to register as a subscriber and publisher for a particular channel or channels. In general, a channel is associated with a publisher at one end and a subscriber at the other end. For example, a particular channel instance for a channel may include a client device 202 publishing messages and a subscribed service 254 receiving the messages. Conversely, the particular channel instance for a channel may include the service 254 publishing messages and a subscribed client device 202 receiving the messages. The particular channel instance may thus be associated with one or more client devices and one or more services. In some examples, a channel instance is associated with one client device 202 and one service 254.

Client devices 204 and 206 are shown in communication with WebSocket endpoint 214. Client devices 204 and 206 may be, for example, subscribed and/or registered as publishers to one or more channels. The client devices 204 and 206 can output variable-length messages including context information and a payload. The context information may include information that identifies the channel and thereby the associated routing information for the channel. The routing subsystem 244 may include components that determine, from the context information, the routing information that identifies at least one consumer of the messages. The routing information may be determined using a set of stream orchestration specifications specified using a polyglot SOL. The set of stream orchestration specifications can each define one or more tasks constituting a “route.” In general, as used herein, a “route” refers generally to a collection of tasks that is determined for each received message and then performed, culminating with outputting the message to at least one downstream consumer identified using the context information. A route can be associated with a channel and the routing of a particular message among a group of client devices and services can be done by a channel instance. The routing subsystem 244 can then route the messages according to the determined routes and output the variable-length message to the determined consumer via WebSocket endpoint 235. For example, system 200 depicts WebSocket endpoint 235 in communication with services 252 and 256.

In an end-to-end example, client device 204 may publish a variable-length message to a channel that includes services 252 and 256. The routing subsystem 244 receives the message at WebSocket endpoint 215 and determines a route to the services 252 and 256 based on the context information in the message. The routing subsystem 244 then processes the message, executing each task in the determined route, sequentially or in parallel, or according to another ordering scheme, until output tasks are reached. Then the routing subsystem 244 outputs the message (or a copy thereof) to the services 252 and 256 via WebSocket endpoint 235.

Turning next to FIG. 3, FIG. 3 depicts another example system 300 for stream orchestration for variable-length message streams, according to some examples of the present disclosure. System 300 shows a detail view of an example implementation of routing subsystem 244 shown in example system 200.

In FIG. 3, routing subsystem 244 has WebSocket endpoints 214 and 234. WebSocket endpoints 214 and 234 can be distinct endpoints (e.g., different hostnames, paths, IP addresses, etc.) or they can be the same endpoint. In the latter case, the WebSocket endpoints 214 and 234 are shown separately in system 300 to illustrate disparate functionality. As shown in FIG. 2, WebSocket endpoints 214 can receive WebSocket frames from client devices. The WebSocket frames can include variable-length messages including context information that implicitly or explicitly specific one or more downstream consumers, such as one or more services.

The WebSocket frames received at WebSocket endpoint 214 may constitute a stream. A stream, as used herein, can refer generally to a continuous flow of WebSocket frames that are received at WebSocket endpoint 214 or output by WebSocket endpoint 234 (or alternatively, received at WebSocket endpoint 234 or output by WebSocket endpoint 214). A stream may be a sequence of WebSocket frames that are logically related and transmitted in a continuous, ordered or orderable manner. For example, a collection of WebSocket frames in a stream may be parts of a larger message or data set that is being sent from one endpoint to another, such as audio data or video data.

The stream of WebSocket frames received at WebSocket endpoint 214 are managed by streaming manager 302 (or streaming manager 330 for WebSocket endpoint 234, respectively). The streaming manager 302 can provide network-layer functions to ensure WebSocket frames are received and processed under various circumstances. For example, the streaming manager 302 can provide buffering to manage varying network speeds, flow control, error handling in response to dropped connections or corrupted data packets, enforcement of security protocols, or other network-layer functions. Streaming managers 302, 330 may be agnostic to the content of the WebSocket frames and configured to provided efficient and error-free transmission and reception of streams.

Incoming WebSocket frames may include a payload that is a variable-length message. The variable-length message can include context information that includes data that collectively identifies a channel instance 310, 320. Each channel instance 310, 320 corresponds to a session that may include one or more client devices and/or one or more servers. The channel instances 310, 320 are created (e.g., class instances instantiated from program code, ephemeral microservices, logically allocated memory, etc.) or selected from among already channel instances 310, 320 upon registration as described above with respect to system 200 in FIG. 2. During registration, a channel is subscribed to or registered as a publisher with respect to, which can cause the channel instance 310, 320 to be created if it does not already exist.

Each channel instance 310, 320 includes a number of tasks. Channel instance 310 includes tasks 312, 314, and 316. Channel instance 320 includes tasks 322, 324, and 326. The tasks depicted in system 300 are shown schematically for illustrative purposes. In practice, the number and configuration of tasks for a channel instance 310 may vary. In particular, the number and configuration of tasks for a particular channel instance 310 can be specified using a set of stream orchestration specifications authored using a polyglot SOL. The tasks 312, 314, and 316 for a particular channel instance 310 may be elements in one or more routes. In general, a route can be one or more of tasks in a channel instance 310. The route for a given variable-length message, through channel instance 310, can be determined using the context information included in the variable-length message using static or dynamic routing techniques as exemplified below.

In some examples, a route includes at least one routing task and an output task. The output task may include a specification of the consumer. For example, the output task may be configured with instructions to output a variable-length to a particular downstream consumer. The downstream consumer may itself have a WebSocket endpoint identified using a URL.

The set of stream orchestration specifications can be specified or written using a polyglot SOL. As used here, a “polyglot SOL” can refer generally to a written, descriptive language for describing stream orchestration specifications that is independent of any particular programming language or underlying implementation. For example, the polyglot SOL can use a programming language-independent data format such as the JavaScript Object Notation (“JSON”) or Extensible Markup Language (“XML”) for describing stream orchestration specifications.

For example, consider a simple stream orchestration specification describing a route with a first task and second task. In this example, the stream orchestration specification can describe a variable-length message being received as input by the first task, processed by the first task, output from the first task to the second task, processed by the second task, and then output from the second task. The first task can be implemented using a first programming language (e.g., Java, Python, or JavaScript) and the second task can be implemented using a second programming language (e.g., Java, C++, or Ruby). The two tasks can be implemented according to an interface specified by documentation or specifications included with the SOL.

The first and second task, implemented using the same or different programming languages, can then be deployed along with the routing subsystem 244 and used for routing. For example, the first and second tasks can be deployed as containerized deployments that can be used by multiple channel instances 310, 320 or may be instantiated for individual channel instances 310, 320 (e.g., instantiated as class or object instances). The first and second task may be implemented to fulfill the contract or interface described in the simple stream orchestration specification. In turn, authors of client applications need only know the details of the simple stream orchestration specification to route messages using the first and second tasks.

The tasks 312, 314, and 316 can be configured to perform any suitable processing, routing, transforming, persisting, or other operation needed during stream orchestration for variable-length message streams. Tasks may include input tasks in which a variable-length message is received following processing by the streaming manager 302, 330. The input task (sometimes referred to as a receive task) may be configured to perform operations such as validating context information or payload, extracting or parsing context information, verifying message or API version compliance, and so on. Tasks may include output tasks in which a variable-length message is output to a streaming manager 302, 330 following routing. The output task may include operations for designating or addressing a downstream consumer such as associating a variable length message with the URL of a service. Another examples of a task type includes a data writing task in which data taken from a variable-length message is persisted to a database or filesystem.

In some examples, the tasks 312, 314, and 316 may include transformation tasks that operate on the variable-length messages or a portion thereof to generate a modified, updated, or edited copy of the variable-length message. These tasks may include transformation tasks such as a text-to-speech task, a speech-to-text task, or a structured data format conversion task.

In various examples, the tasks 312, 314, and 316 may be executed in sequence or in parallel (not shown). FIG. 3 schematically shows tasks 312, 314, and 316 in sequence, but routes may be specified using the SOL with keywords such as “sequence” to indicate tasks that should be executed sequentially or “flow” to indicate tasks that can be executed in parallel. Additional modifiers may be included to facilitate sequential or parallel executions, such as a semaphores or other concurrent execution constructs or mechanisms.

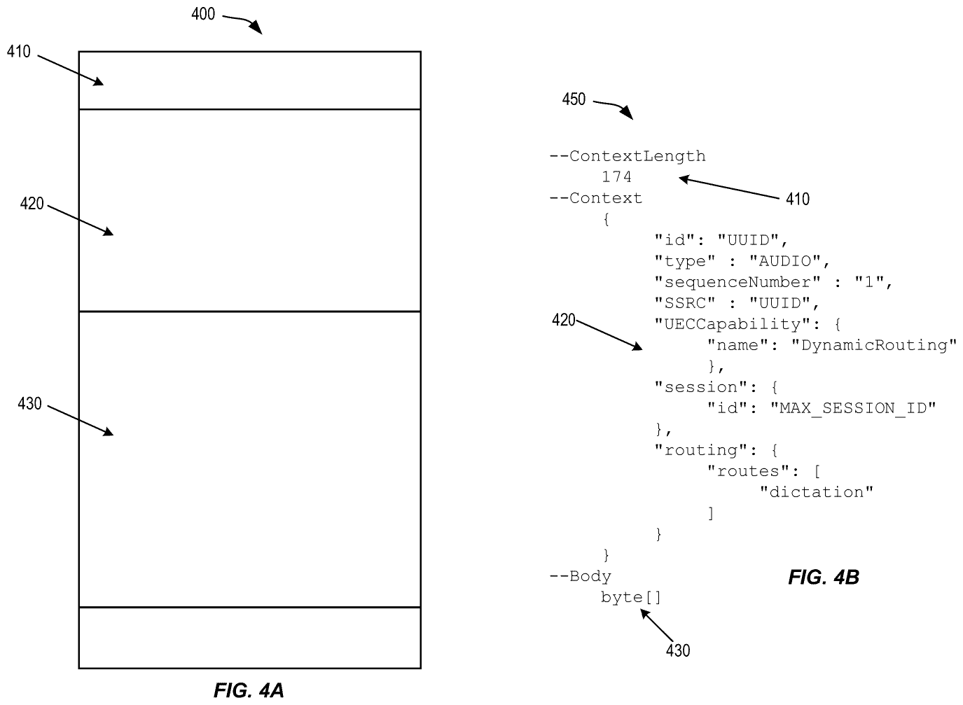

Turning next to FIGS. 4A and 4B, FIGS. 4A and 4B depict example messages 400, 450 that may be used for stream orchestration for variable-length message streams, according to some examples of the present disclosure. The message 400 can be encoded as binary to make it possible to efficiently send it over the network using a compact representation that also avoids the computational overhead of compression. Consequently, the routing subsystem 244 can decode the message 400 to determine the context information 420 that can be used during routing.

To this end, the example variable-length message 400 is shown with three parts. The context information size 410 is includes information about the length of the context information 420. For example, the context information size 410 may be a 4 byte integer that specifies the size, in bytes, of the context information 420. The context information size 410 can thus be used to demarcate the context information 420 and the payload 430. For example, for a 1000 byte variable-length message, including a 900 byte payload 430 and a 96 byte context information 420 section, the context information size 410 may be the 4-byte integer 4 in decimal or 0×00000004 in hexadecimal. The use of a 4-byte integer for the context information size 410 is just an example and not limiting. The maximum value of a signed 4-byte integer is 2,147,483,647, which places a bound on the size of the context information 420. The overall size of the variable-length message including context information 420 and payload 430 is limited only by the constraints of the underlying protocol. For example, the WebSocket protocol may place limits on the variable-length message size. However, in practice, the variable-length messages will be considerably smaller than these limits and limited by network bandwidth or timeouts at other levels of the network stack.

The context information 420 may be, for example, a binary-encoded JSON string. In some examples, the JSON string can be converted to binary using a binary encoding process based on ASCII, UTF-8, UTF-16, or other suitable encoding scheme. In other examples, the context information 420 can be plain text, XML, serialized data, or other suitable format prior to binary encoding. The context information 420 may be decoded in a component of the routing subsystem 244, such as an input or receiving task (e.g., 312 or 322) as described above with respect to FIG. 3.

In some examples, the message 400 is based on a format described using a specification written in a capability interface definition language such as AsyncAPI or OpenAPI. The capability interface definition language can be used to specify, among other things, the associated stream orchestration specification or channel, the URL of the WebSocket endpoint, the data schema and serialization format, authentication and authorization mechanisms, message payload structure, headers or query parameters, event types and handlers, error handling details, versioning, and so on. The capability interface definition language can likewise be used to specify the format of the context information. For example, the context information may be specified to include a number of sections, in which the content of each section is defined using the capability interface definition language. The message specification in the capability interface definition language can be sent from the WebSocket endpoint in response to a suitable query or may be provided as documentation for the stream orchestration subsystem 120.

The payload 430 can include any type of binary data, including encoded text, audio, video, and so on. As described above, the context information size 410 can be used to determine the size of the encoded context information 420. The remainder of the variable-length message 400, after removing the 4-byte context information size 410 and the number of following bytes specified therein (the context information 420), is the payload 430.

Message 450 is an illustration of a populated variable-length message. As explained above, the message 450 may be encoded during transmission and is thus not-human readable. Message 450 is provided to show an example of a variable-length message including data. Message 450 could be a representation of a binary-encoded variable-length message after being decoded for readability. The context information size 410, including the decimal value 174, specifies the number of bytes in the context information 420. The context information 420 is shown as an example JSON object. The context information 420 includes various identifiers and other information that can be used for selecting a route or determining a session. Following the context information 420 is the payload 430, shown as an array of bytes. An array is used to schematically illustrate the payload 430 but the payload 430 can be any collection of textual, encoded binary data, or raw binary data.

Turning next to FIGS. 5A-5C, FIGS. 5A-5C depict example routes 500, 530, 560 that may be used in some implementations of systems for stream orchestration for variable-length message streams, according to some examples of the present disclosure. The routes 500, 530, 560 illustrate three types of routes that may be used in various implementations of stream orchestration for variable-length message streams. Other types of routes may likewise be used in different examples. The example routes 500, 530, 560 are shown with tasks 505-520, including routing task 510. These tasks are shown to illustrate certain concepts. Various examples may have fewer, more, different, or more complex arrangements of tasks.

In FIG. 5A, route 500 illustrates a static route. In a static route, the route can be defined using the SOL. For example, the static route may be defined to include task 505 that receives incoming messages, makes a determination at routing task 510, and then conditionally routes the message to one or both of tasks 515 and/or 520. In some examples, the routing task 510 only has one output in which case the message can be routed without making a determination. The route 500 is static in the sense that it is explicitly specified in the context information of the message. For example, the context information may include a “Route” field or key that specifies a predefined “Dictation” route. As shown, however, even a static route can be conditionally routed, as shown at routing task 510. The route 525 following routing task 510 is thus predetermined and is shown with a solid line.

The routing task 510 may include a specification of a conditional expression. The implementation of the routing task 510 may can evaluate the conditional expression to select the next step or steps in the route. For example, the conditional expression can be evaluated based on the content of the context information. In one example conditional expression, based on a JSON query language, “$.Context.capability== ‘dictation’”, an incoming variable-length message can be routed based on the presence of and/or the value of the “Context.capability” field or key specified in a JSON object contained in the context information. A “JSON query language” may refer generally to an API specification for specifying or identifying locations in a JSON object. If the JSON object includes a top-level object labeled as “Context,” a second-level objected labeled “capability,” and the value “dictation,” the message can be routed to a first task 515. If the value is otherwise, the message can be routed to a second task 520. Conditions may use a variety of APIs provided by the SOL for conditional expressions, including but not limited to, comparison operators (e.g., equals, not equals), logical operators (e.g., and, or), arithmetic functions (e.g., addition, subtraction), string manipulation methods (e.g., contains, substring), date functions (e.g., before, after), type checking (e.g., instance of), collection operations (e.g., is empty, size), pattern matching (e.g., matches), conditional assignment (e.g., ternary operator), bitwise operators (e.g., bitwise and, bitwise or), among others. In some examples, default behaviors for, for instance, when the top-level or second-level objects are not present or malformed, can be defined or implicit in the SOL language specification.

In some examples, the conditional expression can be evaluated by querying a rules engine. For example, routing rules can be externalized to a rules engine so that they can be changed without redeployment. In this case, the static conditional expression can be an expression in a programming language that can be executed at runtime. For example, for a routing task 510 implementation based on the Java programming language the, the conditional expression can be ‘RulesEngine.evaluateExpression (“rule”, data)’. This can invoke a query to a rules engine for a conditional labeled as “rule” with input parameters included in “data.” The “data” may be the context information or a portion thereof.

In FIG. 5B, route 530 shows an example of a dynamic route. Dynamic routing can refer generally to scenarios in which the routing rules are determined based on instructions provided to the routing task 510. Various approaches can be used to effect dynamic routing, two of which are shown in FIGS. 5B and 5C. In route 530, the routes are predefined, similarly to the static route 500 shown in FIG. 5A. However, in route 530, the evaluation made by routing task 510 is based on the context information included in the variable-length message, in contrast to the explicit specification in route 500. In this case, the route 535 following routing task 510 is dynamically determined and may vary between variable-length messages based on the context information included in the variable-length message and is shown with a dashed line.

For example, the route 530 can be used to place control of the route 535 following routing task 510 with the client device outputting a particular message based, for example, a current mode of operation. In this example, multiple client devices connected to the channel instance can route messages based on the mode the client is operating in. For instance, an incoming variable-length message may specify that it is in a “Dictation” mode of operation. In another example, the context information may include a date that determines which version of a service to route the message to. In another example, the context information may include a language that determines which service to route the message to. The route 535 following routing task 510 may be selected by routing task 510. In this example, the client device need not specify or have knowledge of the routes available in the routing subsystem 244.

FIG. 5C shows route 560, another example of a dynamic route. Route 560 depicts a dynamic route based on control events. In a dynamic route based on control events, the route can be explicitly specified in the context information of a variable-length message. The variable-length message containing such a specification may be referred to as a control event message because it can update one or more routes, a change that may impact other client devices. For example, the context information can include a “Route” field that includes one or more definitions of routes using the SOL (e.g., a JSON specification). Based on these stream orchestration specifications, the routing subsystem 244 can update the route in use by one or more corresponding channel instances. The updated route 565 is shown schematically using an alternating dashed-dotted line. Following the update, the routing subsystem can output a broadcast message including information about the updated stream orchestration specification, to alert other client devices to the update. The other client devices can then proceed to route messages statically oy dynamically, using the new route specification.

For example, consider a variable-length message using dynamic routing based on control events received from a client device. In the context information, the message may include a specification of a new route using JSON. For instance, the context information may include a “Route.flow” JSON field that itself includes a JSON route specification in the SOL such as:

```
{
  “type”: “sequence”,
  “tasks”: [
    {
      “type”:“SendStream”,
      “channel”:“objectstore”,
      “stream”:“dictation”
    },
    {
      “type”:“SendStream”,
      “channel”:“ambient”,
      “stream”:“dictation”
    }
  ]
}
```

The existing routes for the corresponding channel instance can be replaced with the routes defined using the SOL excerpt above. To notify all other client devices of the change, the routing subsystem 244 can output a broadcast message including the new route definition. Subsequently, the newly defined routes can be used in concert with static routes or dynamic routes as defined above.

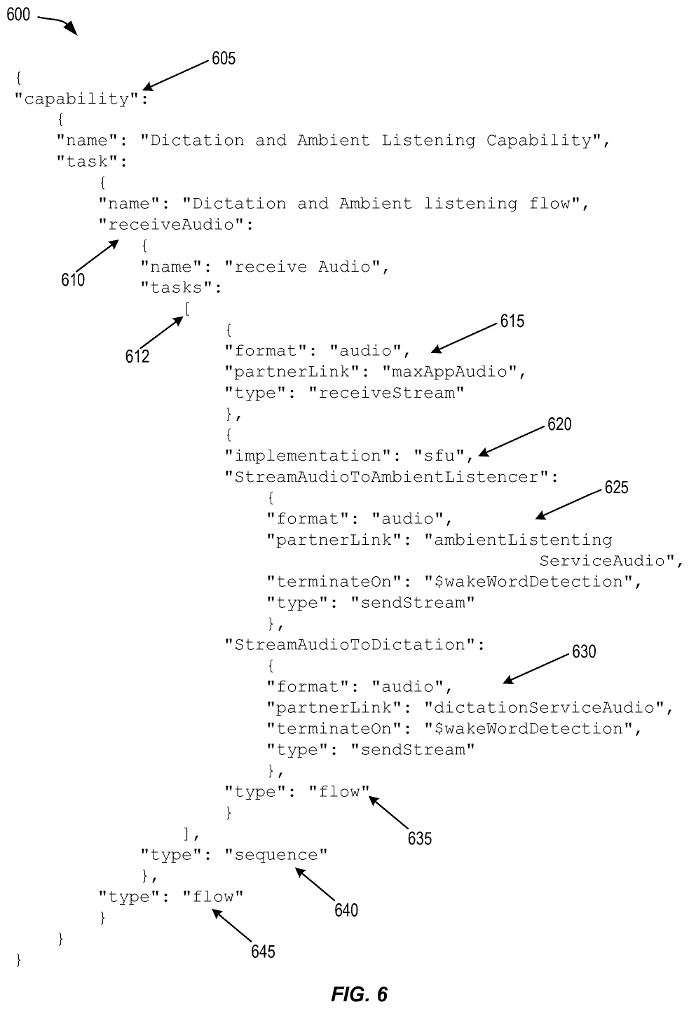

Turning next to FIG. 6, FIG. 6 depicts an example stream orchestration specification 600 using an example SOL for stream orchestration for variable-length message streams, according to some examples of the present disclosure. The example stream orchestration specification 600 is authored using JSON-based SOL, but other semi-structured formats (e.g., XML) or declarative forms of programming languages (e.g., Groovy) could be used to equal effect. As mentioned above, the SOL is a “polyglot SOL,” referring to the implementation-independence of this stream orchestration specification 600. Thus, the example stream orchestration specification 600 declares how the defined route will be behave, but the implementation (e.g., the program code used to cause the behavior) is left up to the system designer.

The stream orchestration specification 600 includes top level object 605. The top level object 605 can correspond to the channel instances 310, 320 described in system 300 of FIG. 3. The word “capability” is a convenient label used in conjunction with top level object 605 to indicate that the following routes making up stream orchestration specification 600 define a capability of the system. For example, capabilities of the routing subsystem may include clinical transcription, an ambient listening, or a clinical automation. A top level object 605 may be associated with a channel, which may in turn be used to instantiate one or more channel instances. In some examples, channels may be referred to as “capabilities” or “skills.”

The stream orchestration specification 600 includes named route 610. The named route 610, like the descriptor “capability,” can provide a human-readable indication of the purpose of the route. In this example, the named route 610 is named “receive Audio,” indicating its purpose to receive incoming variable-length messages with binary audio data payloads. The named route 610 is associated with structural constraint 645. The structural constraint 645 is “flow,” indicating that the named route 610 can be executed in parallel with other tasks, although only one named route 610 is included in top level object 605. The named route 610 includes task array 612, with structural constraint 640 indicating that the tasks enumerated in the task array 612 are to be executed in sequence. The task array 612 includes task 615 and task group 620. Task group 620 includes task 625 and task 630, grouped together using structural constraint 635, “flow,” indicating that the tasks 625, 630 in task group 620 are to be executed in parallel. The example tasks 615, 625, 620 each include a format, a partner link, and a type. The format can correspond to the type of data in the variable-length message payload, which can be used for configuration during execution of certain tasks. The partner link can provide an indication of upstream or downstream tasks with which the specified stream orchestration specification 600 can interface. The type can indicate whether the task is intended for use during input or output of variable-length messages, which can be used for configuration during execution of certain tasks.

The stream orchestration specification 600 is provided to illustrate one example of a stream orchestration specification specified using an example SOL but is not intended to be limiting. Stream orchestration specifications may used other formats, as described above. Furthermore, stream orchestration specifications may include various other language constructs including, but not limited to, variables, functions/subroutines, loops, conditionals (e.g., if-then-else), operators (e.g., addition, subtraction), arrays or other collections, maps or dictionaries, lambda expressions and closures, regular expressions or other pattern matching scheme, recursion, exception handling, object-oriented constructs, concurrency primitives (e.g., threads or locks), asynchronous operations, type casting, string manipulation functions, or generics.

Turning next to FIG. 7, FIG. 7 depicts another example system 700 for stream orchestration for variable-length message streams, according to some examples of the present disclosure. System 700 shows a detail view of an example implementation of stream orchestration subsystem 120 shown in example system 200.

System 700 includes two groups of client devices: client devices 730 and 732 (indicated with diagonal shading) and client devices 740 and 742 (indicated with crosshatch shading). The client devices 730 and 732 are associated with session 705 and channel instance 715. The client devices 740 and 742 are associated with session 710 and channel instance 720.

After establishing a session by subscribing to or registering as a publisher to a channel, the routing subsystem 244 can receive, from a computer system such one of the services 442, 446 a variable-length message. The variable-length message may include context information, as exemplified above in FIGS. 4A-4B. The context information may include a session identifier. for instance, if the context information includes a JSON object, the JSON object may include a top-level field such as “Session” containing a “sessionId” key. For example, the session manager can identify the session for the channel instance (or stream orchestration instance) based on the session identifier by either generating a new session, if it does not already exist, or identifying an already-existing session. For instance, the “sessionId” key can be used to identify a new or existing session.

Generating a new session orchestration instance or channel instance can occur, responsive to receiving the registration information by the session manager 242. The session manager 242 can be configured to generate the stream orchestration instance. For example, the session manager 242 can instantiate a new channel instance using the specified session identifier or other datum. In some examples, for instance, a new class instance can be instantiated using parameters such as the session identifier, known members, and so on. Additional client devices or services can be later added to a given session

The session can have one or more member computer systems. In some examples, the session can include a number of client devices and/or a number of services as well as any other publisher of or subscriber to variable-length messages. In the example system 700, session 705 includes client devices 730 and 732 and session 710 includes client devices 740 and 742. In a one example configuration, a particular session is associated with a number of client devices and one service. The service can then output messages to all the client devices in the session. Thus, in system 700, upon receipt of the variable-length message, the routing subsystem 244 can identify the session using the session manager 242 based in the context information. The routing subsystem 244 can then output the variable-length message to at least one of the member computer systems of the identified session.

In some examples, sessions 705, 710 may have an associated expiration date and/or time. The expiration date and/or time can be used to prevent the accumulation of channel instances to avoid exhausting system resources. For example, the session manager 242 can, upon receipt of a variable-length message that specifies a particular session, determine that the session for the associated channel instance has expired and generate a new session for the stream orchestration instance by, for example, instantiating a new channel instance.

### Illustrative Method(s)

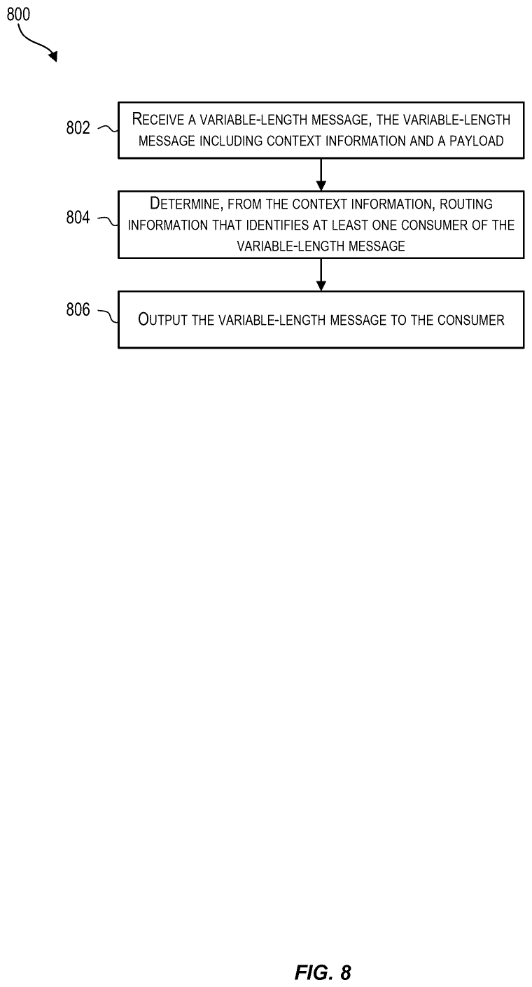

FIG. 8 is a process flow 800 for stream orchestration for variable-length message streams, according to some examples of the present disclosure. The processing depicted in FIG. 8 may be implemented in software (e.g., code, instructions, program) executed by one or more processing units (e.g., processors, cores) of the respective systems, hardware, or combinations thereof. The software may be stored on a non-transitory storage medium (e.g., on a memory device). The method presented in FIG. 8 and described below is intended to be illustrative and non-limiting. Although FIG. 8 depicts the various processing steps occurring in a particular sequence or order, this is not intended to be limiting. In certain alternative embodiments, the steps may be performed in some different order or some steps may also be performed in parallel. In certain embodiments, such as in the embodiments depicted in FIGS. 1-7, the processing depicted in FIG. 8 may be performed by a system (e.g., system 100).

At block 802, a computing system receives a variable-length message, the variable-length message including context information and a payload. The computing system may be, for example, routing subsystem 244 and the variable-length message may be received from a client device and destined for a particular downstream service or services. For instance, the client device may be a healthcare provider's mobile device used for dictation and clinical assistance. The downstream services may be a transcription service and a storage service.

The variable-length message may be encoded in a binary format including three parts: a context information length, the context information, and the payload. The context length can define a demarcation between the context information and the payload. For example, the context length may specified the length of the binary-encoded context information in bytes. By subtracting this length from the length of the variable-length message, the context information and the payload can be separately extracted. In this way, both the context information and the payload can be characterized by a variable-length.

The context information may include one or more key-value pairs. For example, the context information can be a JSON object including a number of top-level keys and associated values, which may themselves be key-value pairs. Other semi-structured or unstructured data formats may be used for the context information such as XML or plain text.

The payload may include, among other things, binary audio data, binary video data, or binary-encoded textual data. In some examples, the variable-length message is included in a message stream including a number of related messages. For example, the related messages may all be generated by a client device following a particular clinical encounter. In some examples, the number of related messages may be ordered or associated with an indicated ordering scheme. For example, the number of related messages may each include a portion of audio or video data which, when aggregated, constitutes an audio or video file or other contiguous unit of data.

In some examples, the variable-length message is received via a WebSocket endpoint. Other configurations are likewise possible. For example, the message could be received via an HTTP request, a RESTful API endpoint, or through a message queue service such as RabbitMQ or Kafka. The endpoint that receives the variable-length may be configured without a maximum message size to accommodate the variable-length messages that may be received. In practice, however, very large payloads (e.g., 10s of megabytes or larger) may cause timeouts or other network congestion. In such cases, a streamed collection of related messages can be used to send smaller portions of the payload that can then be aggregated.

At block 804, the computing system determines, from the context information, routing information that identifies at least one consumer of the variable-length message. For example, the context information may include a top-level object labeled as “Route” or the like that identifies a static route to a particular downstream consumer service, such as the example shown in FIG. 5A. The context information can likewise use the dynamic routes illustrated in FIGS. 5B-5C to route the variable-length message to a particular downstream consumer or consumers.

In some examples, the routing information can be determined from the context information using a set of stream orchestration specifications. Each stream orchestration specification of the set of stream orchestration specifications may include one or more tasks that together define a route. Routes may include at least one routing task. The routing task may determine a number of tasks to direct the received variable-length message to. For example, the routing task may use a conditional expression to determine which of several possible subsequent statically specified routes to direct the received variable-length message to. Routes may include an output task. The output task may include instructions to send the variable-length message to a particular consumer.

In some examples, the set of stream orchestration specifications are specified using a polyglot SOL using a declarative API using a semi-structured format such as JSON or XML. In some examples, a declarative programming language API can be used to equal effect. The “polyglot” SOL can refer generally to independence from the underlying implementation. For example, the SOL can be used to convey to upstream producers or downstream consumers of variable-length messages how the stream orchestration subsystem will behave. At the same time, the underlying behavior can be implemented using any suitable programming language. For example, for a stream orchestration specification defining a route including a first task and second task, the first task can be implemented using a first programming language and the second task can be implemented using a second programming language. The first and second programming languages can be the same or different. Producers and consumers need only be aware of the behavior defined using the SOL.

As mentioned above, the routing information can be classified broadly as static or dynamic, although this should not be construed as limiting. In static routing schemes, the context information of the variable-length message include an explicit specification of a route that corresponds to a particular stream orchestration specification. For instance, the context information may include a “Route” field or key with a string literal named route specified, such as “sendAudio.” The named route “sendAudio” can correspond to a correspondingly named route defined in the set of stream orchestration specifications. The routing subsystem 244 can identify the named route using exact or partial string matching and direct to the variable-length message to the named route.

In some examples, the stream orchestration specification may include a routing task. The routing task may precede a number of groups of tasks, each group being a subsequent possible route following the routing task. The routing task can be configured to evaluate a conditional expression which determined which of the group or groups of tasks will receive the variable-length message. The conditional expression can be, for example, written in a JSON query language such as JSONPath and evaluated in given the context information as input in the routing task. In some examples, the conditional expression can be evaluated by querying a rules engine. Using a rules engine can be advantageous in that conditional expressions can be modified without redeploying the stream orchestration subsystem. In contrast, changes to the fixed conditional expressions included in the set of stream orchestration specifications may require redeployment of the stream orchestration subsystem or a portion thereof.

In some examples, the one or more tasks of can be executed in sequence or in parallel. For example, a “flow” keyword in the stream orchestration specification may be used to indicate tasks that can be performed in parallel. A “sequence” keyword can be used to indicate tasks that can be performed in sequence. In some examples, certain tasks may have dependencies and therefore must be performed in sequence. The “flow” and “sequence” keywords, or equivalent, can be associated with all tasks in a stream orchestration specification, groups of tasks (e.g., a JSON array of tasks), individual tasks, and so on.

In addition the input, output, and routing, the one or more tasks included in a stream orchestration specification may perform various functions. For example, the one or more tasks can include a transformation task such as a text-to-speech task, a speech-to-text task, or a structured data format conversion task. These and other example can be used to replace or update the context information, the payload, or both during routing. In some examples, the one or more tasks may include a data writing task for persisting task the context information, the payload, or both to a database or filesystem. Other non-limiting examples of functions that can be performed by tasks include compression, encryption/decryption, data validation, error handling, logging, notification, filtering, aggregation, and so on.

In dynamic routing schemes, the context information is used to determine the route. In one example, the route may be determined using a value or values from the context information to determine which of a number of possible statically defined routes to use. This form of dynamic routing is similar to the static routing scheme described above, except that the route is not explicitly specified. In another example, the route can be dynamically specified using a stream orchestration specification included in the context information. In this case, the context information may include a complete route specification written using the SOL. The dynamic stream orchestration specifications can be identified in the context information and updated in the corresponding channel instance. At the same time, the routing subsystem 244 can output a broadcast message to all client devices including information about the updated route. The updated route can then be used as the target of static or dynamic routes, as described above.

The consumers may include, for example, clinical transcription services, ambient listening services, clinical automation services, and so on. These services, upon receipt of the variable-length message, may cause an update to a clinical record. For example, these services may update patient information, treatment plans, or diagnostic data in near-real-time.

Examples of clinical transcription services may include transcribing of provider-patient consultation notes, converting audio recordings of procedures into written reports, transforming dictated radiology findings into structured text reports, and so on. Ambient listening services might involve continuously capturing and analyzing ambient speech during a provider-patient encounter to detect verbal suggestions or commands given by healthcare providers to update patient records. Clinical automation services may include automatically updating patient records with vital signs data from client devices, logging medication administration events into patient records, adjusting and updating patient appointment schedules, importing lab test results into patient records, or generating alerts for healthcare providers when certain critical thresholds are reached.

At block 806, the computing system outputs the variable-length message to the consumer. For example, the route determined using the routing information may include an output task. The output task may be configured to output the variable-length message, or a copied or transformed version thereof, to a designated downstream consumer. The output task can be created or instantiated, along with the channel instance, with configuration information such as the IP address or hostname of the downstream consumer service or other network location. In some examples, the variable-length message is added to a stream and output as part of a message stream.

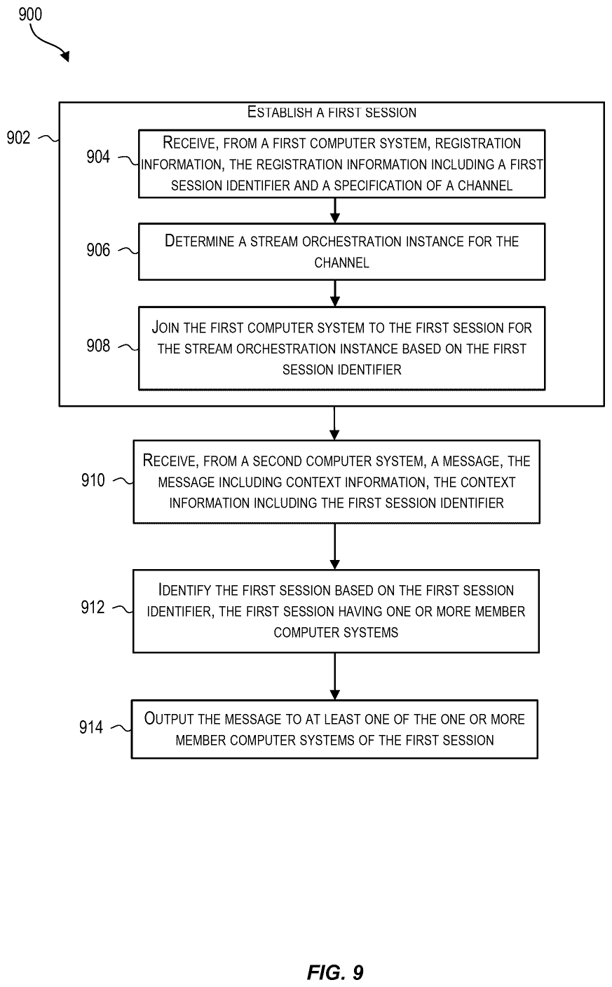

FIG. 9 is a process flow 900 for stream orchestration for variable-length message streams, according to some examples of the present disclosure. The processing depicted in FIG. 9 may be implemented in software (e.g., code, instructions, program) executed by one or more processing units (e.g., processors, cores) of the respective systems, hardware, or combinations thereof. The software may be stored on a non-transitory storage medium (e.g., on a memory device). The method presented in FIG. 9 and described below is intended to be illustrative and non-limiting. Although FIG. 9 depicts the various processing steps occurring in a particular sequence or order, this is not intended to be limiting. In certain alternative embodiments, the steps may be performed in some different order or some steps may also be performed in parallel. In certain embodiments, such as in the embodiments depicted in FIGS. 1-7, the processing depicted in FIG. 9 may be performed by a system (e.g., system 100).

At block 902, a computing system such as the routing subsystem 244 establishes a first session. Establishing the first session includes block 904. At block 904, the computing system receives, from a first computer system such as a client device 730, registration information, the registration information including a first session identifier and a specification of a channel. For example, the first computer system can output a POST or GET request to a REST API endpoint 212 including a resource for registering with a channel and/or establishing a session. Since these operations are related, the same REST resource may be used. In some examples, one REST resource can be used for subscribing or registering as a publisher with channels and a different REST resource can be used for establishing a session. In the latter case, a session may be associated with more than one channel. In some examples, the first computer system can likewise output a WebSocket authentication message to a WebSocket endpoint 214 including the registration information. For instance, the first computer system can initiate a handshake with WebSocket endpoint 214, establishing a near-real-time persistent connection that can enable bidirectional communication. Once the handshake is completed, the first computer system can send the authentication message including the registration information for subscribing or registering as a publisher with various channels and establishing a session.

Establishing the first session also includes block 906. At block 906, the computing system determines a stream orchestration instance for the channel, also referred to as a channel instance. When a new session is created, such as session 705, a corresponding channel instance 715 can be created. The corresponding channel instance 715 can alternatively be referred to as a capability or skill or as an instance thereof. If the channel instance 715, the computing system can instead identify it. The computing system may maintain an index of sessions and associated channel indexes so that existing sessions and corresponding channel instances can be quickly identified.

Establishing the first session next includes block 908. At block 908, the computing system joins the first computer system to the first session for the stream orchestration instance based on the first session identifier. For example, the computing system can access the index of sessions and associated channel indexes. The first computer system can be added to the session so that when the session or channel index is later used, the first computer system will receive messages, as described below.

At block 910, the computing system receives, from a second computer system, a message, the message including context information, the context information including the first session identifier. For example, the second computer system may be a service 446 outputting a message to the session 705 associated with the client device 730. The first session identifier can be extracted from a field or key of the context information such as “sessionId.” The first session identifier may be an alphanumeric string corresponding to a known existing session, if not known, can be an indication to create a new session.

At block 912, the computing system identifies the first session based on the first session identifier, the first session having one or more member computer systems. For example, the routing subsystem 244 can receive the message and the extracted session identifier. The session identifier can be input to the session manager 242 to receive a reference (e.g., a network location or name) of a channel instance associated with the session identifier. For instance, the session manager 242 can use the index of sessions and associated channel indexes to identify the session associated with the session identifier and create a reference thereto. Alternatively, the session manager 242 may create a session as described above and create a reference to the newly created channel instance.

At block 914, the computing system outputs the message to at least one of the one or more member computer systems of the first session. The first session can include identifying information about all of the client devices 730, 732 joined to it. The routing subsystem 244 can output the message to the joined client devices 730, 732 by making a suitable change or update to the context information of each message. Then, a downstream task, such as an output task, can output the message to each respective client device 730, 732. For example, the routing subsystem 244 can designate an IP address or hostname of each of the joined client devices 730, 732 to which an output task can route each message.

The process 900 describes a message being routed from the second computer system to one or more client devices based on membership in the first session. In general, message routing using the session management process 900 can be unidirectional or bi-directional. For example, consider a second message received from the first computer system, also associated with the first session. For some session configurations, the second message may be output to the one or more members of the first session including the second computer system. In this scenario, the message routing configuration of the first session is bi-directional. In some examples, the second message may be output to the one or more members of the first session but not including the second computer system. In this scenario, the message routing configuration of the first session is unidirectional. In this case, messages can be sent from the second computer system to the first, but not from the first computer system to the second computer system. The designation of a session as unidirectional or bi-directional can be made during session creation using, for example, REST API endpoint 212 or WebSocket endpoint 214.

As noted above, infrastructure as a service (IaaS) is one particular type of cloud computing that can be used in association with stream orchestration for variable-length message streams. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance. Some examples of cloud infrastructure that can support deployments of systems or components for stream orchestration for variable-length message streams are now described.

### Examples Of Cloud Infrastructure

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 10 is a block diagram 1000 illustrating an example pattern of an IaaS architecture, according to at least one embodiment. Service operators 1002 can be communicatively coupled to a secure host tenancy 1004 that can include a virtual cloud network (VCN) 1006 and a secure host subnet 1008. In some examples, the service operators 1002 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1006 and/or the Internet.

The VCN 1006 can include a local peering gateway (LPG) 1010 that can be communicatively coupled to a secure shell (SSH) VCN 1012 via an LPG 1010 contained in the SSH VCN 1012. The SSH VCN 1012 can include an SSH subnet 1014, and the SSH VCN 1012 can be communicatively coupled to a control plane VCN 1016 via the LPG 1010 contained in the control plane VCN 1016. Also, the SSH VCN 1012 can be communicatively coupled to a data plane VCN 1018 via an LPG 1010. The control plane VCN 1016 and the data plane VCN 1018 can be contained in a service tenancy 1019 that can be owned and/or operated by the IaaS provider.

The control plane VCN 1016 can include a control plane demilitarized zone (DMZ) tier 1020 that acts as a perimeter network (e.g., portions of a corporate network between the corporate intranet and external networks). The DMZ-based servers may have restricted responsibilities and help keep breaches contained. Additionally, the DMZ tier 1020 can include one or more load balancer (LB) subnet(s) 1022, a control plane app tier 1024 that can include app subnet(s) 1026, a control plane data tier 1028 that can include database (DB) subnet(s) 1030 (e.g., frontend DB subnet(s) and/or backend DB subnet(s)). The LB subnet(s) 1022 contained in the control plane DMZ tier 1020 can be communicatively coupled to the app subnet(s) 1026 contained in the control plane app tier 1024 and an Internet gateway 1034 that can be contained in the control plane VCN 1016, and the app subnet(s) 1026 can be communicatively coupled to the DB subnet(s) 1030 contained in the control plane data tier 1028 and a service gateway 1036 and a network address translation (NAT) gateway 1038. The control plane VCN 1016 can include the service gateway 1036 and the NAT gateway 1038.

The control plane VCN 1016 can include a data plane mirror app tier 1040 that can include app subnet(s) 1026. The app subnet(s) 1026 contained in the data plane mirror app tier 1040 can include a virtual network interface controller (VNIC) 1042 that can execute a compute instance 1044. The compute instance 1044 can communicatively couple the app subnet(s) 1026 of the data plane mirror app tier 1040 to app subnet(s) 1026 that can be contained in a data plane app tier 1046.

The data plane VCN 1018 can include the data plane app tier 1046, a data plane DMZ tier 1048, and a data plane data tier 1050. The data plane DMZ tier 1048 can include LB subnet(s) 1022 that can be communicatively coupled to the app subnet(s) 1026 of the data plane app tier 1046 and the Internet gateway 1034 of the data plane VCN 1018. The app subnet(s) 1026 can be communicatively coupled to the service gateway 1036 of the data plane VCN 1018 and the NAT gateway 1038 of the data plane VCN 1018. The data plane data tier 1050 can also include the DB subnet(s) 1030 that can be communicatively coupled to the app subnet(s) 1026 of the data plane app tier 1046.

The Internet gateway 1034 of the control plane VCN 1016 and of the data plane VCN 1018 can be communicatively coupled to a metadata management service 1052 that can be communicatively coupled to public Internet 1054. Public Internet 1054 can be communicatively coupled to the NAT gateway 1038 of the control plane VCN 1016 and of the data plane VCN 1018. The service gateway 1036 of the control plane VCN 1016 and of the data plane VCN 1018 can be communicatively coupled to cloud services 1056.

In some examples, the service gateway 1036 of the control plane VCN 1016 or of the data plane VCN 1018 can make application programming interface (API) calls to cloud services 1056 without going through public Internet 1054. The API calls to cloud services 1056 from the service gateway 1036 can be one-way: the service gateway 1036 can make API calls to cloud services 1056, and cloud services 1056 can send requested data to the service gateway 1036. But, cloud services 1056 may not initiate API calls to the service gateway 1036.

In some examples, the secure host tenancy 1004 can be directly connected to the service tenancy 1019, which may be otherwise isolated. The secure host subnet 1008 can communicate with the SSH subnet 1014 through an LPG 1010 that may enable two-way communication over an otherwise isolated system. Connecting the secure host subnet 1008 to the SSH subnet 1014 may give the secure host subnet 1008 access to other entities within the service tenancy 1019.

The control plane VCN 1016 may allow users of the service tenancy 1019 to set up or otherwise provision desired resources. Desired resources provisioned in the control plane VCN 1016 may be deployed or otherwise used in the data plane VCN 1018. In some examples, the control plane VCN 1016 can be isolated from the data plane VCN 1018, and the data plane mirror app tier 1040 of the control plane VCN 1016 can communicate with the data plane app tier 1046 of the data plane VCN 1018 via VNICs 1042 that can be contained in the data plane mirror app tier 1040 and the data plane app tier 1046.

In some examples, users of the system, or customers, can make requests, for example create, read, update, or delete (CRUD) operations, through public Internet 1054 that can communicate the requests to the metadata management service 1052. The metadata management service 1052 can communicate the request to the control plane VCN 1016 through the Internet gateway 1034. The request can be received by the LB subnet(s) 1022 contained in the control plane DMZ tier 1020. The LB subnet(s) 1022 may determine that the request is valid, and in response to this determination, the LB subnet(s) 1022 can transmit the request to app subnet(s) 1026 contained in the control plane app tier 1024. If the request is validated and requires a call to public Internet 1054, the call to public Internet 1054 may be transmitted to the NAT gateway 1038 that can make the call to public Internet 1054. Metadata that may be desired to be stored by the request can be stored in the DB subnet(s) 1030.

In some examples, the data plane mirror app tier 1040 can facilitate direct communication between the control plane VCN 1016 and the data plane VCN 1018. For example, changes, updates, or other suitable modifications to configuration may be desired to be applied to the resources contained in the data plane VCN 1018. Via a VNIC 1042, the control plane VCN 1016 can directly communicate with, and can thereby execute the changes, updates, or other suitable modifications to configuration to, resources contained in the data plane VCN 1018.

In some embodiments, the control plane VCN 1016 and the data plane VCN 1018 can be contained in the service tenancy 1019. In this case, the user, or the customer, of the system may not own or operate either the control plane VCN 1016 or the data plane VCN 1018. Instead, the IaaS provider may own or operate the control plane VCN 1016 and the data plane VCN 1018, both of which may be contained in the service tenancy 1019. This embodiment can enable isolation of networks that may prevent users or customers from interacting with other users′, or other customers′, resources. Also, this embodiment may allow users or customers of the system to store databases privately without needing to rely on public Internet 1054, which may not have a desired level of threat prevention, for storage.

In other embodiments, the LB subnet(s) 1022 contained in the control plane VCN 1016 can be configured to receive a signal from the service gateway 1036. In this embodiment, the control plane VCN 1016 and the data plane VCN 1018 may be configured to be called by a customer of the IaaS provider without calling public Internet 1054. Customers of the IaaS provider may desire this embodiment since database(s) that the customers use may be controlled by the IaaS provider and may be stored on the service tenancy 1019, which may be isolated from public Internet 1054.

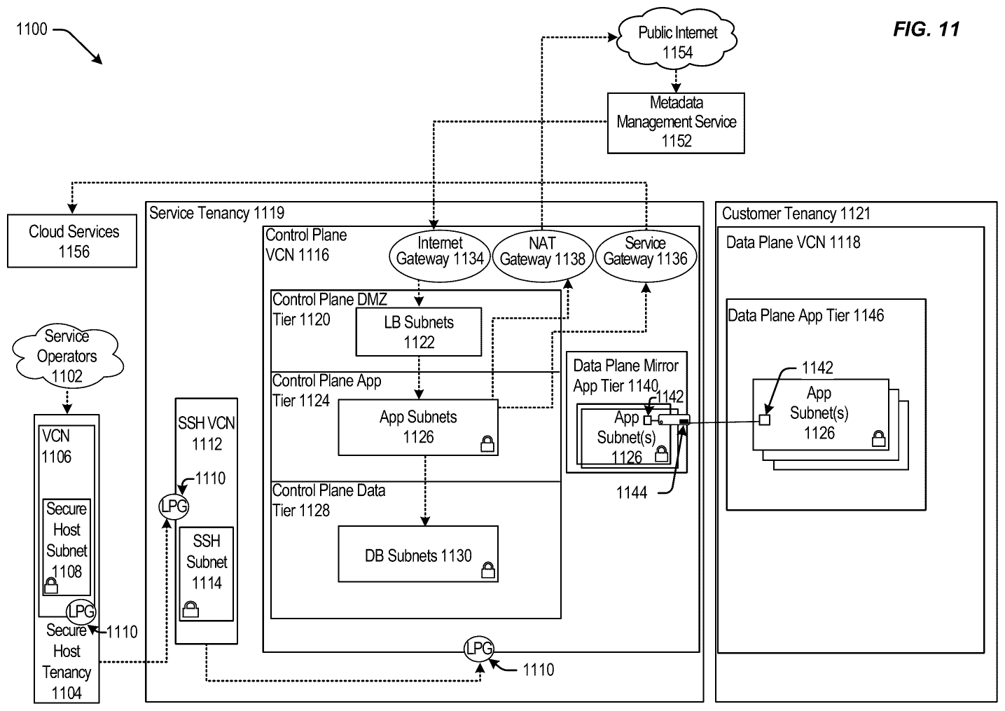

FIG. 11 is a block diagram 1100 illustrating another example pattern of an IaaS architecture, according to at least one embodiment. Service operators 1102 (e.g., service operators 1002 of FIG. 10) can be communicatively coupled to a secure host tenancy 1104 (e.g., the secure host tenancy 1004 of FIG. 10) that can include a virtual cloud network (VCN) 1106 (e.g., the VCN 1006 of FIG. 10) and a secure host subnet 1108 (e.g., the secure host subnet 1008 of FIG. 10). The VCN 1106 can include a local peering gateway (LPG) 1110 (e.g., the LPG 1010 of FIG. 10) that can be communicatively coupled to a secure shell (SSH) VCN 1112 (e.g., the SSH VCN 1012 of FIG. 10) via an LPG 1010 contained in the SSH VCN 1112. The SSH VCN 1112 can include an SSH subnet 1114 (e.g., the SSH subnet 1014 of FIG. 10), and the SSH VCN 1112 can be communicatively coupled to a control plane VCN 1116 (e.g., the control plane VCN 1016 of FIG. 10) via an LPG 1110 contained in the control plane VCN 1116. The control plane VCN 1116 can be contained in a service tenancy 1119 (e.g., the service tenancy 1019 of FIG. 10), and the data plane VCN 1118 (e.g., the data plane VCN 1018 of FIG. 10) can be contained in a customer tenancy 1121 that may be owned or operated by users, or customers, of the system.

The control plane VCN 1116 can include a control plane DMZ tier 1120 (e.g., the control plane DMZ tier 1020 of FIG. 10) that can include LB subnet(s) 1122 (e.g., LB subnet(s) 1022 of FIG. 10), a control plane app tier 1124 (e.g., the control plane app tier 1024 of FIG. 10) that can include app subnet(s) 1126 (e.g., app subnet(s) 1026 of FIG. 10), a control plane data tier 1128 (e.g., the control plane data tier 1028 of FIG. 10) that can include database (DB) subnet(s) 1130 (e.g., similar to DB subnet(s) 1030 of FIG. 10). The LB subnet(s) 1122 contained in the control plane DMZ tier 1120 can be communicatively coupled to the app subnet(s) 1126 contained in the control plane app tier 1124 and an Internet gateway 1134 (e.g., the Internet gateway 1034 of FIG. 10) that can be contained in the control plane VCN 1116, and the app subnet(s) 1126 can be communicatively coupled to the DB subnet(s) 1130 contained in the control plane data tier 1128 and a service gateway 1136 (e.g., the service gateway 1036 of FIG. 10) and a network address translation (NAT) gateway 1138 (e.g., the NAT gateway 1038 of FIG. 10). The control plane VCN 1116 can include the service gateway 1136 and the NAT gateway 1138.

The control plane VCN 1116 can include a data plane mirror app tier 1140 (e.g., the data plane mirror app tier 1040 of FIG. 10) that can include app subnet(s) 1126. The app subnet(s) 1126 contained in the data plane mirror app tier 1140 can include a virtual network interface controller (VNIC) 1142 (e.g., the VNIC of 1042) that can execute a compute instance 1144 (e.g., similar to the compute instance 1044 of FIG. 10). The compute instance 1144 can facilitate communication between the app subnet(s) 1126 of the data plane mirror app tier 1140 and the app subnet(s) 1126 that can be contained in a data plane app tier 1146 (e.g., the data plane app tier 1046 of FIG. 10) via the VNIC 1142 contained in the data plane mirror app tier 1140 and the VNIC 1142 contained in the data plane app tier 1146.

The Internet gateway 1134 contained in the control plane VCN 1116 can be communicatively coupled to a metadata management service 1152 (e.g., the metadata management service 1052 of FIG. 10) that can be communicatively coupled to public Internet 1154 (e.g., public Internet 1054 of FIG. 10). Public Internet 1154 can be communicatively coupled to the NAT gateway 1138 contained in the control plane VCN 1116. The service gateway 1136 contained in the control plane VCN 1116 can be communicatively coupled to cloud services 1156 (e.g., cloud services 1056 of FIG. 10).

In some examples, the data plane VCN 1118 can be contained in the customer tenancy 1121. In this case, the IaaS provider may provide the control plane VCN 1116 for each customer, and the IaaS provider may, for each customer, set up a unique compute instance 1144 that is contained in the service tenancy 1119. Each compute instance 1144 may allow communication between the control plane VCN 1116, contained in the service tenancy 1119, and the data plane VCN 1118 that is contained in the customer tenancy 1121. The compute instance 1144 may allow resources, that are provisioned in the control plane VCN 1116 that is contained in the service tenancy 1119, to be deployed or otherwise used in the data plane VCN 1118 that is contained in the customer tenancy 1121.

In other examples, the customer of the IaaS provider may have databases that live in the customer tenancy 1121. In this example, the control plane VCN 1116 can include the data plane mirror app tier 1140 that can include app subnet(s) 1126. The data plane mirror app tier 1140 can reside in the data plane VCN 1118, but the data plane mirror app tier 1140 may not live in the data plane VCN 1118. That is, the data plane mirror app tier 1140 may have access to the customer tenancy 1121, but the data plane mirror app tier 1140 may not exist in the data plane VCN 1118 or be owned or operated by the customer of the IaaS provider. The data plane mirror app tier 1140 may be configured to make calls to the data plane VCN 1118 but may not be configured to make calls to any entity contained in the control plane VCN 1116. The customer may desire to deploy or otherwise use resources in the data plane VCN 1118 that are provisioned in the control plane VCN 1116, and the data plane mirror app tier 1140 can facilitate the desired deployment, or other usage of resources, of the customer.

In some embodiments, the customer of the IaaS provider can apply filters to the data plane VCN 1118. In this embodiment, the customer can determine what the data plane VCN 1118 can access, and the customer may restrict access to public Internet 1154 from the data plane VCN 1118. The IaaS provider may not be able to apply filters or otherwise control access of the data plane VCN 1118 to any outside networks or databases. Applying filters and controls by the customer onto the data plane VCN 1118, contained in the customer tenancy 1121, can help isolate the data plane VCN 1118 from other customers and from public Internet 1154.

In some embodiments, cloud services 1156 can be called by the service gateway 1136 to access services that may not exist on public Internet 1154, on the control plane VCN 1116, or on the data plane VCN 1118. The connection between cloud services 1156 and the control plane VCN 1116 or the data plane VCN 1118 may not be live or continuous. Cloud services 1156 may exist on a different network owned or operated by the IaaS provider. Cloud services 1156 may be configured to receive calls from the service gateway 1136 and may be configured to not receive calls from public Internet 1154. Some cloud services 1156 may be isolated from other cloud services 1156, and the control plane VCN 1116 may be isolated from cloud services 1156 that may not be in the same region as the control plane VCN 1116. For example, the control plane VCN 1116 may be located in “Region 1,” and cloud service “Deployment 10,” may be located in Region 1 and in “Region 2.” If a call to Deployment 10 is made by the service gateway 1136 contained in the control plane VCN 1116 located in Region 1, the call may be transmitted to Deployment 10 in Region 1. In this example, the control plane VCN 1116, or Deployment 10 in Region 1, may not be communicatively coupled to, or otherwise in communication with, Deployment 10 in Region 2.

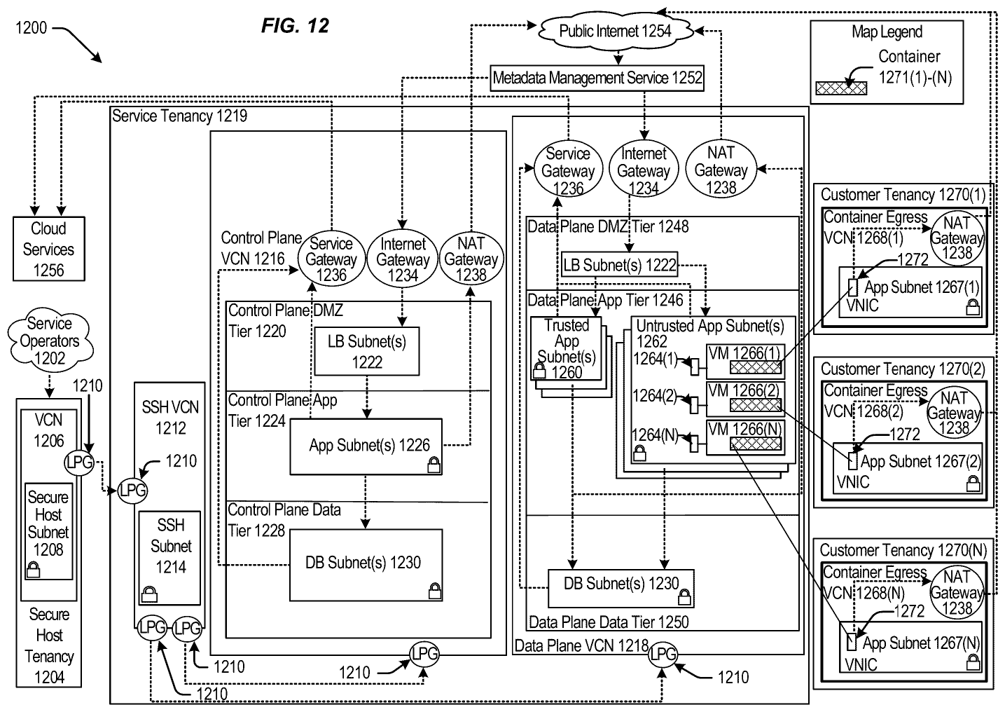

FIG. 12 is a block diagram 1200 illustrating another example pattern of an IaaS architecture, according to at least one embodiment. Service operators 1202 (e.g., service operators 1002 of FIG. 10) can be communicatively coupled to a secure host tenancy 1204 (e.g., the secure host tenancy 1004 of FIG. 10) that can include a virtual cloud network (VCN) 1206 (e.g., the VCN 1006 of FIG. 10) and a secure host subnet 1208 (e.g., the secure host subnet 1008 of FIG. 10). The VCN 1206 can include an LPG 1210 (e.g., the LPG 1010 of FIG. 10) that can be communicatively coupled to an SSH VCN 1212 (e.g., the SSH VCN 1012 of FIG. 10) via an LPG 1210 contained in the SSH VCN 1212. The SSH VCN 1212 can include an SSH subnet 1214 (e.g., the SSH subnet 1014 of FIG. 10), and the SSH VCN 1212 can be communicatively coupled to a control plane VCN 1216 (e.g., the control plane VCN 1016 of FIG. 10) via an LPG 1210 contained in the control plane VCN 1216 and to a data plane VCN 1218 (e.g., the data plane 1018 of FIG. 10) via an LPG 1210 contained in the data plane VCN 1218. The control plane VCN 1216 and the data plane VCN 1218 can be contained in a service tenancy 1219 (e.g., the service tenancy 1019 of FIG. 10).

The control plane VCN 1216 can include a control plane DMZ tier 1220 (e.g., the control plane DMZ tier 1020 of FIG. 10) that can include load balancer (LB) subnet(s) 1222 (e.g., LB subnet(s) 1022 of FIG. 10), a control plane app tier 1224 (e.g., the control plane app tier 1024 of FIG. 10) that can include app subnet(s) 1226 (e.g., similar to app subnet(s) 1026 of FIG. 10), a control plane data tier 1228 (e.g., the control plane data tier 1028 of FIG. 10) that can include DB subnet(s) 1230. The LB subnet(s) 1222 contained in the control plane DMZ tier 1220 can be communicatively coupled to the app subnet(s) 1226 contained in the control plane app tier 1224 and to an Internet gateway 1234 (e.g., the Internet gateway 1034 of FIG. 10) that can be contained in the control plane VCN 1216, and the app subnet(s) 1226 can be communicatively coupled to the DB subnet(s) 1230 contained in the control plane data tier 1228 and to a service gateway 1236 (e.g., the service gateway of FIG. 10) and a network address translation (NAT) gateway 1238 (e.g., the NAT gateway 1038 of FIG. 10). The control plane VCN 1216 can include the service gateway 1236 and the NAT gateway 1238.

The data plane VCN 1218 can include a data plane app tier 1246 (e.g., the data plane app tier 1046 of FIG. 10), a data plane DMZ tier 1248 (e.g., the data plane DMZ tier 1048 of FIG. 10), and a data plane data tier 1250 (e.g., the data plane data tier 1050 of FIG. 10). The data plane DMZ tier 1248 can include LB subnet(s) 1222 that can be communicatively coupled to trusted app subnet(s) 1260 and untrusted app subnet(s) 1262 of the data plane app tier 1246 and the Internet gateway 1234 contained in the data plane VCN 1218. The trusted app subnet(s) 1260 can be communicatively coupled to the service gateway 1236 contained in the data plane VCN 1218, the NAT gateway 1238 contained in the data plane VCN 1218, and DB subnet(s) 1230 contained in the data plane data tier 1250. The untrusted app subnet(s) 1262 can be communicatively coupled to the service gateway 1236 contained in the data plane VCN 1218 and DB subnet(s) 1230 contained in the data plane data tier 1250. The data plane data tier 1250 can include DB subnet(s) 1230 that can be communicatively coupled to the service gateway 1236 contained in the data plane VCN 1218.

The untrusted app subnet(s) 1262 can include one or more primary VNICs 1264 (1)-(N) that can be communicatively coupled to tenant virtual machines (VMs) 1266 (1)-(N). Each tenant VM 1266 (1)-(N) can be communicatively coupled to a respective app subnet 1267 (1)-(N) that can be contained in respective container egress VCNs 1268 (1)-(N) that can be contained in respective customer tenancies 1270 (1)-(N). Respective secondary VNICs 1272 (1)-(N) can facilitate communication between the untrusted app subnet(s) 1262 contained in the data plane VCN 1218 and the app subnet contained in the container egress VCNs 1268 (1)-(N). Each container egress VCNs 1268 (1)-(N) can include a NAT gateway 1238 that can be communicatively coupled to public Internet 1254 (e.g., public Internet 1054 of FIG. 10).

The Internet gateway 1234 contained in the control plane VCN 1216 and contained in the data plane VCN 1218 can be communicatively coupled to a metadata management service 1252 (e.g., the metadata management system 1052 of FIG. 10) that can be communicatively coupled to public Internet 1254. Public Internet 1254 can be communicatively coupled to the NAT gateway 1238 contained in the control plane VCN 1216 and contained in the data plane VCN 1218. The service gateway 1236 contained in the control plane VCN 1216 and contained in the data plane VCN 1218 can be communicatively coupled to cloud services 1256.

In some embodiments, the data plane VCN 1218 can be integrated with customer tenancies 1270. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1246. Code to run the function may be executed in the VMs 1266 (1)-(N), and the code may not be configured to run anywhere else on the data plane VCN 1218. Each VM 1266 (1)-(N) may be connected to one customer tenancy 1270. Respective containers 1271 (1)-(N) contained in the VMs 1266 (1)-(N) may be configured to run the code. In this case, there can be a dual isolation (e.g., the containers 1271 (1)-(N) running code, where the containers 1271 (1)-(N) may be contained in at least the VM 1266 (1)-(N) that are contained in the untrusted app subnet(s) 1262), which may help prevent incorrect or otherwise undesirable code from damaging the network of the IaaS provider or from damaging a network of a different customer. The containers 1271 (1)-(N) may be communicatively coupled to the customer tenancy 1270 and may be configured to transmit or receive data from the customer tenancy 1270. The containers 1271 (1)-(N) may not be configured to transmit or receive data from any other entity in the data plane VCN 1218. Upon completion of running the code, the IaaS provider may kill or otherwise dispose of the containers 1271 (1)-(N).

In some embodiments, the trusted app subnet(s) 1260 may run code that may be owned or operated by the IaaS provider. In this embodiment, the trusted app subnet(s) 1260 may be communicatively coupled to the DB subnet(s) 1230 and be configured to execute CRUD operations in the DB subnet(s) 1230. The untrusted app subnet(s) 1262 may be communicatively coupled to the DB subnet(s) 1230, but in this embodiment, the untrusted app subnet(s) may be configured to execute read operations in the DB subnet(s) 1230. The containers 1271 (1)-(N) that can be contained in the VM 1266 (1)-(N) of each customer and that may run code from the customer may not be communicatively coupled with the DB subnet(s) 1230.

In other embodiments, the control plane VCN 1216 and the data plane VCN 1218 may not be directly communicatively coupled. In this embodiment, there may be no direct communication between the control plane VCN 1216 and the data plane VCN 1218. However, communication can occur indirectly through at least one method. An LPG 1210 may be established by the IaaS provider that can facilitate communication between the control plane VCN 1216 and the data plane VCN 1218. In another example, the control plane VCN 1216 or the data plane VCN 1218 can make a call to cloud services 1256 via the service gateway 1236. For example, a call to cloud services 1256 from the control plane VCN 1216 can include a request for a service that can communicate with the data plane VCN 1218.

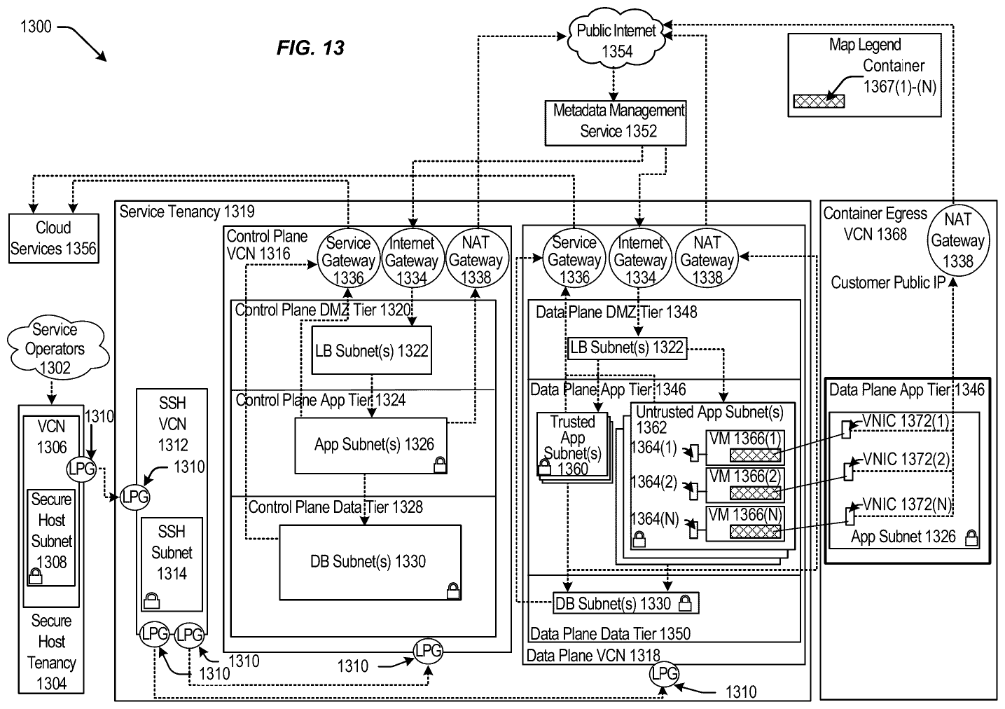

FIG. 13 is a block diagram 1300 illustrating another example pattern of an IaaS architecture, according to at least one embodiment. Service operators 1302 (e.g., service operators 1002 of FIG. 10) can be communicatively coupled to a secure host tenancy 1304 (e.g., the secure host tenancy 1004 of FIG. 10) that can include a virtual cloud network (VCN) 1306 (e.g., the VCN 1006 of FIG. 10) and a secure host subnet 1308 (e.g., the secure host subnet 1008 of FIG. 10). The VCN 1306 can include an LPG 1310 (e.g., the LPG 1010 of FIG. 10) that can be communicatively coupled to an SSH VCN 1312 (e.g., the SSH VCN 1012 of FIG. 10) via an LPG 1310 contained in the SSH VCN 1312. The SSH VCN 1312 can include an SSH subnet 1314 (e.g., the SSH subnet 1014 of FIG. 10), and the SSH VCN 1312 can be communicatively coupled to a control plane VCN 1316 (e.g., the control plane VCN 1016 of FIG. 10) via an LPG 1310 contained in the control plane VCN 1316 and to a data plane VCN 1318 (e.g., the data plane 1018 of FIG. 10) via an LPG 1310 contained in the data plane VCN 1318. The control plane VCN 1316 and the data plane VCN 1318 can be contained in a service tenancy 1319 (e.g., the service tenancy 1019 of FIG. 10).

The control plane VCN 1316 can include a control plane DMZ tier 1320 (e.g., the control plane DMZ tier 1020 of FIG. 10) that can include LB subnet(s) 1322 (e.g., LB subnet(s) 1022 of FIG. 10), a control plane app tier 1324 (e.g., the control plane app tier 1024 of FIG. 10) that can include app subnet(s) 1326 (e.g., app subnet(s) 1026 of FIG. 10), a control plane data tier 1328 (e.g., the control plane data tier 1028 of FIG. 10) that can include DB subnet(s) 1330 (e.g., DB subnet(s) 1230 of FIG. 12). The LB subnet(s) 1322 contained in the control plane DMZ tier 1320 can be communicatively coupled to the app subnet(s) 1326 contained in the control plane app tier 1324 and to an Internet gateway 1334 (e.g., the Internet gateway 1034 of FIG. 10) that can be contained in the control plane VCN 1316, and the app subnet(s) 1326 can be communicatively coupled to the DB subnet(s) 1330 contained in the control plane data tier 1328 and to a service gateway 1336 (e.g., the service gateway of FIG. 10) and a network address translation (NAT) gateway 1338 (e.g., the NAT gateway 1038 of FIG. 10). The control plane VCN 1316 can include the service gateway 1336 and the NAT gateway 1338.

The data plane VCN 1318 can include a data plane app tier 1346 (e.g., the data plane app tier 1046 of FIG. 10), a data plane DMZ tier 1348 (e.g., the data plane DMZ tier 1048 of FIG. 10), and a data plane data tier 1350 (e.g., the data plane data tier 1050 of FIG. 10). The data plane DMZ tier 1348 can include LB subnet(s) 1322 that can be communicatively coupled to trusted app subnet(s) 1360 (e.g., trusted app subnet(s) 1260 of FIG. 12) and untrusted app subnet(s) 1362 (e.g., untrusted app subnet(s) 1262 of FIG. 12) of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include primary VNICs 1364 (1)-(N) that can be communicatively coupled to tenant virtual machines (VMs) 1366 (1)-(N) residing within the untrusted app subnet(s) 1362. Each tenant VM 1366 (1)-(N) can run code in a respective container 1367 (1)-(N), and be communicatively coupled to an app subnet 1326 that can be contained in a data plane app tier 1346 that can be contained in a container egress VCN 1368. Respective secondary VNICs 1372 (1)-(N) can facilitate communication between the untrusted app subnet(s) 1362 contained in the data plane VCN 1318 and the app subnet contained in the container egress VCN 1368. The container egress VCN can include a NAT gateway 1338 that can be communicatively coupled to public Internet 1354 (e.g., public Internet 1054 of FIG. 10).

The Internet gateway 1334 contained in the control plane VCN 1316 and contained in the data plane VCN 1318 can be communicatively coupled to a metadata management service 1352 (e.g., the metadata management system 1052 of FIG. 10) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively coupled to cloud services 1356.

In some examples, the pattern illustrated by the architecture of block diagram 1300 of FIG. 13 may be considered an exception to the pattern illustrated by the architecture of block diagram 1200 of FIG. 12 and may be desirable for a customer of the IaaS provider if the IaaS provider cannot directly communicate with the customer (e.g., a disconnected region). The respective containers 1367 (1)-(N) that are contained in the VMs 1366 (1)-(N) for each customer can be accessed in real-time by the customer. The containers 1367 (1)-(N) may be configured to make calls to respective secondary VNICs 1372 (1)-(N) contained in app subnet(s) 1326 of the data plane app tier 1346 that can be contained in the container egress VCN 1368. The secondary VNICs 1372 (1)-(N) can transmit the calls to the NAT gateway 1338 that may transmit the calls to public Internet 1354. In this example, the containers 1367 (1)-(N) that can be accessed in real-time by the customer can be isolated from the control plane VCN 1316 and can be isolated from other entities contained in the data plane VCN 1318. The containers 1367 (1)-(N) may also be isolated from resources from other customers.

In other examples, the customer can use the containers 1367 (1)-(N) to call cloud services 1356. In this example, the customer may run code in the containers 1367 (1)-(N) that requests a service from cloud services 1356. The containers 1367 (1)-(N) can transmit this request to the secondary VNICs 1372 (1)-(N) that can transmit the request to the NAT gateway that can transmit the request to public Internet 1354. Public Internet 1354 can transmit the request to LB subnet(s) 1322 contained in the control plane VCN 1316 via the Internet gateway 1334. In response to determining the request is valid, the LB subnet(s) can transmit the request to app subnet(s) 1326 that can transmit the request to cloud services 1356 via the service gateway 1336.

It should be appreciated that IaaS architectures 1000, 1100, 1200, 1300 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

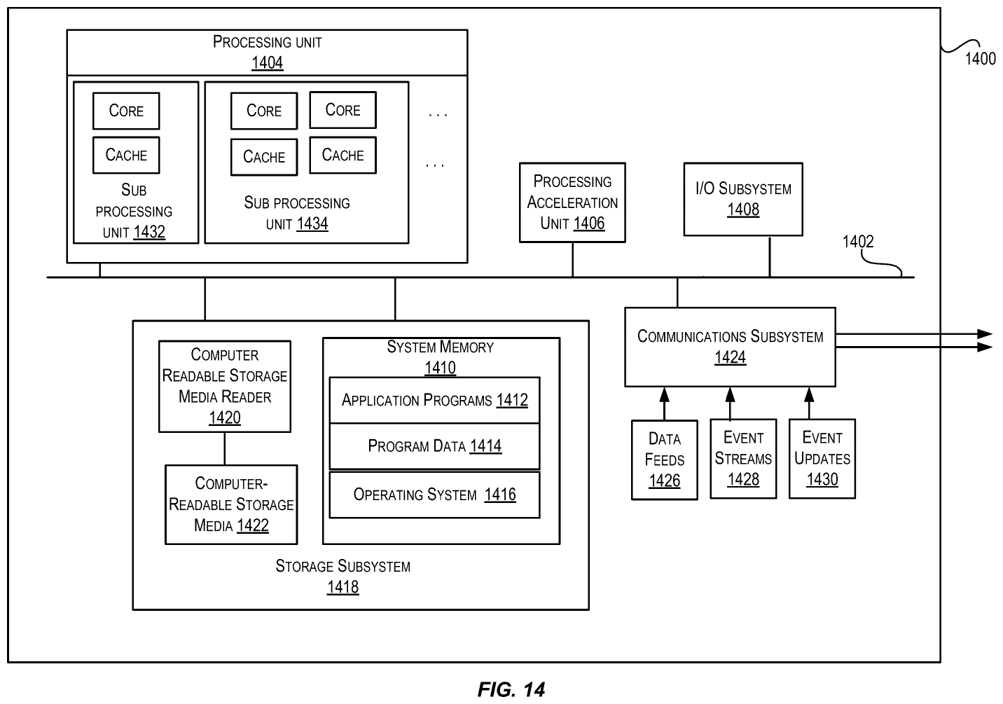

FIG. 14 illustrates an example computer system 1400, in which various embodiments may be implemented. The system 1400 may be used to implement any of the computer systems described above. As shown in the figure, computer system 1400 includes a processing unit 1404 that communicates with a number of peripheral subsystems via a bus subsystem 1402. These peripheral subsystems may include a processing acceleration unit 1406, an I/O subsystem 1408, a storage subsystem 1418 and a communications subsystem 1424. Storage subsystem 1418 includes tangible computer-readable storage media 1422 and a system memory 1410.

Bus subsystem 1402 provides a mechanism for letting the various components and subsystems of computer system 1400 communicate with each other as intended. Although bus subsystem 1402 is shown schematically as a single bus, alternative embodiments of the bus subsystem may utilize multiple buses. Bus subsystem 1402 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1404, which can be implemented as one or more integrated circuits (e.g., a conventional microprocessor or microcontroller), controls the operation of computer system 1400. One or more processors may be included in processing unit 1404. These processors may include single core or multicore processors. In certain embodiments, processing unit 1404 may be implemented as one or more independent processing units 1432 and/or 1434 with single or multicore processors included in each processing unit. In other embodiments, processing unit 1404 may also be implemented as a quad-core processing unit formed by integrating two dual-core processors into a single chip.

In various embodiments, processing unit 1404 can execute a variety of programs in response to program code and can maintain multiple concurrently executing programs or processes. At any given time, some or all of the program code to be executed can be resident in processor(s) 1404 and/or in storage subsystem 1418. Through suitable programming, processor(s) 1404 can provide various functionalities described above. Computer system 1400 may additionally include a processing acceleration unit 1406, which can include a digital signal processor (DSP), a special-purpose processor, and/or the like.

I/O subsystem 1408 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1400 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1400 may comprise a storage subsystem 1418 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1404 provide the functionality described above. Storage subsystem 1418 may also provide a repository for storing data used in accordance with the present disclosure.

As depicted in the example in FIG. 14, storage subsystem 1418 can include various components including a system memory 1410, computer-readable storage media 1422, and a computer readable storage media reader 1420. System memory 1410 may store program instructions that are loadable and executable by processing unit 1404. System memory 1410 may also store data that is used during the execution of the instructions and/or data that is generated during the execution of the program instructions. Various different kinds of programs may be loaded into system memory 1410 including but not limited to client applications, Web browsers, mid-tier applications, relational database management systems (RDBMS), virtual machines, containers, etc.

System memory 1410 may also store an operating system 1416. Examples of operating system 1416 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1400 executes one or more virtual machines, the virtual machines along with their guest operating systems (GOSs) may be loaded into system memory 1410 and executed by one or more processors or cores of processing unit 1404.

System memory 1410 can come in different configurations depending upon the type of computer system 1400. For example, system memory 1410 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1410 may include a basic input/output system (BIOS) containing basic routines that help to transfer information between elements within computer system 1400, such as during start-up.

Computer-readable storage media 1422 may represent remote, local, fixed, and/or removable storage devices plus storage media for temporarily and/or more permanently containing, storing, computer-readable information for use by computer system 1400 including instructions executable by processing unit 1404 of computer system 1400.

Computer-readable storage media 1422 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1422 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1422 may include, but is not limited to, Zip® drives, flash memory cards, universal serial bus (USB) flash drives, secure digital (SD) cards, DVD disks, digital video tape, and the like. Computer-readable storage media 1422 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1400.

Machine-readable instructions executable by one or more processors or cores of processing unit 1404 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1424 provides an interface to other computer systems and networks. Communications subsystem 1424 serves as an interface for receiving data from and transmitting data to other systems from computer system 1400. For example, communications subsystem 1424 may enable computer system 1400 to connect to one or more devices via the Internet. In some embodiments communications subsystem 1424 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 1424 can provide wired network connectivity (e.g., Ethernet) in addition to or instead of a wireless interface.

In some embodiments, communications subsystem 1424 may also receive input communication in the form of structured and/or unstructured data feeds 1426, event streams 1428, event updates 1430, and the like on behalf of one or more users who may use computer system 1400.

By way of example, communications subsystem 1424 may be configured to receive data feeds 1426 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1424 may also be configured to receive data in the form of continuous data streams, which may include event streams 1428 of real-time events and/or event updates 1430, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1424 may also be configured to output the structured and/or unstructured data feeds 1426, event streams 1428, event updates 1430, and the like to one or more databases that may be in communication with one or more streaming data source computers coupled to computer system 1400.

Computer system 1400 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1400 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method, comprising:
establishing a first session, wherein establishing the first session comprises:
receiving, from a first computer system, registration information, the registration information including a first session identifier and a specification of a channel,
determining a stream orchestration instance for the channel, and
joining the first computer system to the first session for the stream orchestration instance based on the first session identifier;
receiving, from a second computer system, a message, the message including context information, the context information including the first session identifier;
identifying the first session based on the first session identifier, the first session having one or more member computer systems; and
outputting the message to at least one of the one or more member computer systems of the first session.

2. The computer-implemented method of claim 1, wherein:
the message is a variable-length message further including a payload;
the message is encoded in a binary format; and
the context information comprises a context length and one or more key-value pairs, the context length defining a demarcation between the context information and the payload, the context information and the payload both characterized by a variable-length.

3. The computer-implemented method of claim 1, further comprising:
determining, from the context information, routing information including a specification of the at least one of the one or more member computer systems, the routing information based on a set of stream orchestration specifications, wherein each stream orchestration specification of the set of stream orchestration specifications includes one or more tasks defining a route, including at least one routing task and an output task, the output task including the specification of the at least one of the one or more member computer systems.

4. The computer-implemented method of claim 3, wherein:
the routing information includes one or more first tasks defining a first route; and
the routing information is determined based on a specification of the first route that is included in the context information.

5. The computer-implemented method of claim 1, wherein:
at least one of the one or more member computer systems is a clinical transcription service, an ambient listening service, or a clinical automation service; and
outputting the message to the at least one of the one or more member computer systems causes an update to a clinical record.

6. The computer-implemented method of claim 1, wherein joining the first computer system to the first session for the stream orchestration instance based on the first session identifier comprises generating a new session for the stream orchestration instance.

7. The computer-implemented method of claim 1, wherein joining the first computer system to the first session for the stream orchestration instance based on the first session identifier comprises joining an existing session for the stream orchestration instance.

8. The computer-implemented method of claim 1, further comprising:
determining that the first session for the stream orchestration instance has expired; and
generating a new session for the stream orchestration instance.

9. The computer-implemented method of claim 1, further comprising, responsive to receiving the registration information, generating the stream orchestration instance.

10. The computer-implemented method of claim 1, wherein the at least one of the one or more member computer systems includes the first computer system and further comprising:
receiving, from the first computer system, a second message, the second message including second context information, the second context information including the first session identifier;
identifying the first session based on the first session identifier; and
outputting the message to the at least one of the one or more member computer systems of the first session including the second computer system.

11. The computer-implemented method of claim 1, further comprising:
receiving, from the first computer system, a second message, the second message including second context information, the second context information including the first session identifier;
identifying the first session based on the first session identifier; and
outputting the message to the at least one of the one or more member computer systems of the first session not including the second computer system.

12. The computer-implemented method of claim 1, wherein the message is based on a format described using a specification written in a capability interface definition language.

13. The computer-implemented method of claim 12, wherein the channel is defined using the capability interface definition language.

14. The computer-implemented method of claim 12, wherein:
the capability interface definition language is AsyncAPI; and
the message is received using a WebSocket endpoint.

15. The computer-implemented method of claim 12, wherein the context information includes a plurality of sections, wherein content of each section is defined in the specification using the capability interface definition language.

16. A system comprising:
one or more processors; and
one or more computer-readable media storing instructions which, when executed by the one or more processors, cause the system to perform operations comprising:
establishing a session, comprising:
receiving, from a first computer system, registration information, the registration information including a first session identifier and a specification of a channel;
determining a stream orchestration instance for the channel; and
joining the first computer system to a first session for the stream orchestration instance based on the first session identifier;
receiving, from a second computer system, a message, the message including context information, the context information including the first session identifier; and
identifying the first session based on the first session identifier, the first session having one or more member computer systems; and
outputting the message to at least one of the one or more member computer systems of the first session.

17. The system of claim 16, wherein:
the message is a variable-length message further including a payload;
the message is encoded in a binary format;
the context information comprises a context length and one or more key-value pairs, the context length defining a demarcation between the context information and the payload, the context information and the payload both characterized by a variable-length; and
the operations further comprise:
determining, from the context information, routing information including a specification of the at least one of the one or more member computer systems, the routing information based on a set of stream orchestration specifications, wherein each stream orchestration specification of the set of stream orchestration specifications includes one or more tasks defining a route, including at least one routing task and an output task, the output task including the specification of the at least one of the one or more member computer systems.

18. The system of claim 17, wherein:
the message is based on a format described using a specification written in a capability interface definition language; and
the message is received using a WebSocket endpoint.

19. One or more non-transitory computer-readable media storing instructions which, when executed by one or more processors, cause a system to perform operations comprising:
establishing a session, comprising:
receiving, from a first computer system, registration information, the registration information including a first session identifier and a specification of a channel;
determining a stream orchestration instance for the channel; and
joining the first computer system to a first session for the stream orchestration instance based on the first session identifier;
receiving, from a second computer system, a message, the message including context information, the context information including the first session identifier; and
identifying the first session based on the first session identifier, the first session having one or more member computer systems; and
outputting the message to at least one of the one or more member computer systems of the first session.

20. The non-transitory computer-readable media of claim 19, wherein
the message is a variable-length message further including a payload;
the message is encoded in a binary format;
the context information comprises a context length and one or more key-value pairs, the context length defining a demarcation between the context information and the payload, the context information and the payload both characterized by a variable-length; and
the operations further comprise:
determining, from the context information, routing information including a specification of the at least one of the one or more member computer systems, the routing information based on a set of stream orchestration specifications, wherein each stream orchestration specification of the set of stream orchestration specifications includes one or more tasks defining a route, including at least one routing task and an output task, the output task including the specification of the at least one of the one or more member computer systems.
